(19) United States
(12) Patent Application Publication
(10) Pub. No.: US 2021/0338749 A1
(43) Pub. Date: Nov. 4, 2021

(54) METHODS OF TREATMENT OF INFLAMMATORY CONDITIONS AND ASSOCIATED INFECTIONS

(71) Applicant: Servatus Ltd., Queensland (AU)

(72) Inventor: Wayne FINLAYSON, Queensland (AU)

(21) Appl. No.: 17/284,207

(22) PCT Filed: Oct. 10, 2019
(86) PCT No.: PCT/AU2019/051092

§ 371 Date: Apr. 9, 2021

(30) Foreign Application Priority Data
Oct. 10, 2018 (AU) 2018903822
Oct. 10, 2018 (AU) 2018903823
Apr. 5, 2019 (AU) 2019901175
Apr. 5, 2019 (AU) 2019901176

(51) Int. Cl. A61K 35/747; A23L 33/135; A61P 31/04; A61P 1/04; A61P 19/02; A61P 17/10; A61P 29/00; A61P 1/12; A61P 31/10; A61P 17/06

(52) U.S. Cl. CPC A61K 35/747; A23L 33/135; A61P 31/04; A61P 1/04; A61P 17/06; A61P 17/10; A61P 29/00; A61P 1/12; A61P 31/10; A61P 19/02

(57) ABSTRACT

Provided herein are methods for treating or preventing inflammation, inflammatory conditions and autoimmune conditions, optionally of the gastrointestinal tract, urinary tract, skin, nails or joints. Also provided are methods of treating or preventing bacterial infections, typically infections associated with inflammation, inflammatory conditions and autoimmune conditions of the gastrointestinal tract, urinary tract, skin, nails or joints. The methods of the present disclosure comprise administering to a subject in need a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

### FIELD OF THE ART

The present disclosure relates generally to methods for the treatment or prevention of inflammation and of inflammatory and autoimmune conditions, and for the treatment of infections associated with such inflammation, and inflammatory and autoimmune conditions. Typically the inflammation and inflammatory and autoimmune conditions are of the gastrointestinal tract, urinary tract, skin, nails or joints, typically being conditions associated with, or caused by pathogenic infections. The present disclosure also relates to the promotion of wound healing. The methods of the present disclosure comprise the administration of compositions comprising one or more microorganisms, or culture supernatants or cell free filtrates derived from culture media in which the one or more microorganisms has been cultured.

### BACKGROUND

Inflammation is a normal response mechanism assisting in protecting the body from infection and injury. However abnormal or uncontrolled inflammatory responses can result in the development of acute or chronic inflammatory and autoimmune disorders or conditions. In particular, infections caused by viruses, fungi and pathogenic bacteria can trigger excessive and persistent inflammatory responses in a variety of tissues, such as of the gastrointestinal tract, joints, skin and the urinary tract, leading to deleterious acute inflammation and acute inflammatory conditions. These are also a significant risk factor in the development of chronic inflammatory and autoimmune conditions. Chronic inflammatory and autoimmune conditions can be debilitating and cause enormous discomfort and pain to sufferers. Moreover, such conditions are increasing in prevalence as populations around the world age.

Inflammatory bowel disease is a complex chronic idiopathic condition characterized by the alteration and dysregulation of immune response towards commensal microbiota of the gastrointestinal tract. There are two main subtypes of inflammatory bowel disease, Crohn's disease and ulcerative colitis. Crohn's disease can occur at any point along the lower gastrointestinal tract while ulcerative colitis is mostly limited to the colon and can predispose individuals to colitis-associated cancer, typically colorectal cancer.

The etiology of inflammatory bowel disease is still somewhat unclear, although dysbiosis in the gut microbiota characterized by an overwhelming increase in pathogenic strains and a decrease in beneficial or commensal resident microorganisms in the gut is increasingly implicated. Among the implicated microorganisms, E coli strains are heavily represented in biopsy samples collected from patients with Crohn's disease and ulcerative colitis. These strains have been shown to adhere and invade gut epithelium and are commonly referred to as adherent and invasive E. coli (AIEC). AIEC have been shown to be closely associated with the mucosal membrane and play a key role in the pathogenesis of Crohn's disease. AIEC requires the ability to adhere and colonise intestinal cell surfaces in order to instigate and exacerbate chronic inflammation in susceptible individuals. E. coli strains isolated from active Crohn's disease patients have shown an ability to adhere to Coco-2 cells, which mimics the intestinal epithelium. AIEC also invade the intestinal epithelial cells where they persist, replicate and drive proinflammatory activities. Their invasive abilities have also been linked to an increase in the severity of ileal inflammatory disease.

Steroids have been the primary therapeutic anti-inflammatory agent relied upon for many decades. More recently non-steroidal anti-inflammatory drugs (NSAIDs) have begun to be commonly employed to manage or treat inflammation. However, the continued use of such agents comes with significant disadvantages and side effects. For example, associated with continued NSAID use are significant side effects including stomach ulcers and bleeding. Additionally, it is well known that NSAIDs produce lesions in the gastrointestinal tract, depending on the length of the treatment and on the type of drug. This problem is of particular importance in cases where the therapy must be protracted for a long time, such as in the treatment of chronic inflammatory disorders where long term treatment is needed to manage the inflammatory state and associated pain.

There is a continuing need for the development of new and improved therapeutic options for the treatment of inflammation and inflammatory and autoimmune conditions.

Wound healing is a complex and precise biological process involving a number of biological factors and requiring a finely tuned balance between different physiological processes. While inflammation is part of the wound healing process, the sensitivity of the process is dependent on the balance between a variety of molecules and pathways and can be easily disrupted. Thus, mechanisms of wound healing and tissue repair are often inadequate and incomplete. For example, chronic wounds such as pressure sores and diabetic foot ulcers, fail to heal appropriately, and are becoming an increasing problem worldwide. Wound healing can also be substantially impaired in the elderly, in cancer patients after chemotherapy or radiation treatments and in individuals suffering from severe burns. The lesions caused by conditions such as Crohn's disease or osteoarthritis are also characterized by slow and deficient healing.

### SUMMARY OF THE DISCLOSURE

A first aspect of the present disclosure provides a method for treating or preventing inflammation, or an inflammatory or autoimmune condition, or one or more symptoms associated therewith, in a subject, comprising administering to the subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

The inflammation may be inflammation of the gastrointestinal tract, urinary tract, skin, nails or joints. The gastrointestinal inflammation may be of the upper gastrointestinal tract such as the mouth or throat, or of the lower gastrointestinal tract, such as the stomach, small intestine or large intestine. The inflammatory or autoimmune condition may be an inflammatory or autoimmune condition of the gastrointestinal tract, urinary tract, skin, nails or joints.

In particular embodiments the inflammation is induced by or associated with an infection. The inflammation may be acute inflammation or chronic inflammation. In exemplary embodiments the pathogen causing the infection is a bacteria.

A second aspect of the present disclosure provides a method for treating or preventing a condition of the gastrointestinal tract, urinary tract, skin, nails or joints in a subject, comprising administering to the subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured, wherein the conditions is associated with inflammation of the gastrointestinal tract, urinary tract, skin, nails or joints and/or wherein the condition is caused by or associated with an infection of the gastrointestinal tract, urinary tract, skin, nails or joints.

In accordance with the first and second aspects, the gastrointestinal inflammation or the condition of the gastrointestinal tract may be, or be associated with, gastritis, gastroenteritis, an inflammatory bowel disease or irritable bowel syndrome. The inflammatory bowel disease may be, for example, colitis, such as ulcerative colitis or Crohn's disease. The ulcerative colitis may be chronic ulcerative colitis. Alternatively, the gastrointestinal inflammation or the condition of the gastrointestinal tract may be, or may be associated with, a condition of the mouth or throat including, for example, gingivitis, tonsillitis and pharyngitis such as streptococcal pharyngitis.

The method may be employed to treat or prevent one or more symptoms of a gastrointestinal infection, such as food poisoning. The gastrointestinal infection may be a bacterial, viral or parasitic infection. The at least one symptom may be abdominal pain, abdominal cramping, abdominal bloating, diarrhoea, poor stool consistency, or faecal blood presence.

In accordance with the first and second aspects, the condition of the urinary tract may be, or be associated with, cystitis, urethritis, pyelonephritis, asymptomatic bacteriuria or a catheter-associated urinary tract infection.

In accordance with the first and second aspects, the condition of the skin or nails may be, or be associated with, psoriasis, dermatitis, eczema, rosacea, acne, ichtyosis, tinea or other skin or nail condition characterized by or associated with inflammation, plaques, skin lesions and/or infection. The infection may be caused by, for example, a pathogenic bacteria or fungus.

In accordance with the first and second aspects, the condition of the joints may be, or be associated with, arthritis. The arthritis may be, for example, rheumatoid arthritis or osteoarthritis.

In accordance with the first and second aspects, the condition may be caused by, or associated with an infection. Such conditions include, for example, gastritis, gastroenteritis, mastitis, gingivitis, pharyngitis such as streptococcal pharyngitis (Strep throat) and conditions of the skin and nails. The infection may be a bacterial, viral, fungal or parasitic infection.

A third aspect of the present disclosure provides a method for treating or preventing a bacterial infection of the gastrointestinal tract, urinary tract, skin, nails or joints, comprising administering to a subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

Typically in accordance with the third aspect the bacterial infection causes, induces or is otherwise associated with inflammation, or an inflammatory or autoimmune condition.

A fourth aspect of the present disclosure provides a method for treating or preventing an inflammatory or autoimmune condition of the gastrointestinal tract, optionally wherein the condition is caused by or associated with an infection, comprising administering to a subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

The condition may be an acute condition or a chronic condition. In exemplary embodiments the condition may be selected from an inflammatory bowel disease, gastritis, gastroenteritis and gingivitis. The inflammatory bowel disease may be colitis. The colitis may be, for example, ulcerative colitis or Crohn's disease. The ulcerative colitis may be chronic ulcerative colitis.

In an exemplary embodiment, the subject is administered a combination of L. paracasei, L. buchneri and L. zeae. In a further exemplary embodiment, the subject is administered a combination of L. diolivorans, L. parafarraginis and L. buchneri.

A fifth aspect of the present disclosure provides a method for treating or preventing irritable bowel syndrome or an inflammatory bowel disease, comprising administering to a subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

The inflammatory bowel disease may be, for example, ulcerative colitis or Crohn's disease. The ulcerative colitis may be chronic ulcerative colitis.

In an exemplary embodiment, the subject is administered a combination of L. paracasei, L. buchneri and L. zeae. In a further exemplary embodiment, the subject is administered a combination of L. diolivorans, L. parafarraginis and L. buchneri.

A sixth aspect of the present disclosure provides a method for treating or preventing a bacterial infection of the gastrointestinal tract, comprising administering to a subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

The infection may be associated with adhesion and/or invasion of the gastrointestinal epithelium by the bacteria causing the infection. The infection may cause, induce or be otherwise associated with inflammation of the gastrointestinal tract or an inflammatory or autoimmune condition of the gastrointestinal tract.

A seventh aspect of the present disclosure provides a method for inhibiting or preventing adhesion of a bacterial pathogen to the gastrointestinal mucosa in a subject, comprising administering to the subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

In exemplary embodiments the gastrointestinal mucosa comprises the epithelial lining of the stomach, duodenum or the lower gastrointestinal tract. Typically the bacterial pathogen is a pathogen that colonises the gastrointestinal tract.

An eighth aspect of the present disclosure provides a method for inhibiting or preventing invasion of gastrointestinal epithelial cells of a subject by a bacterial pathogen, comprising administering to the subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

In exemplary embodiments the gastrointestinal epithelial cells comprise the epithelial cells of the stomach, duodenum or the lower gastrointestinal tract. Typically the bacterial pathogen is a pathogen that colonises the gastrointestinal tract.

A further aspect of the present disclosure provides a method for promoting wound healing in a subject, comprising administering to the subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

In accordance with the above aspects and embodiments, the method may comprise the administration of a combination of two, three, four, five or all six of said Lactobacillus species, or culture supernatants or cell free filtrates derived from culture media in which two, three, four, five or all six of said Lactobacillus have been cultured. The combination may represent a synergistic combination.

In accordance with the above aspects and embodiments, the Lactobacillus may be administered, for example, orally, sublingually or topically.

In particular embodiments the Lactobacillus species, culture supernatant(s) or cell free filtrate(s) may be administered in the form of a pharmaceutically acceptable composition, or a food or beverage.

In accordance with the above aspects and embodiments, the method may further comprise the administration of one or more additional microorganisms or other therapeutic agents.

In accordance with the above aspects and embodiments, the method may comprise administering to the subject a microbial biotherapeutic composition of the Lactobacillus species. The microbial biotherapeutic composition may be administered in the form of, for example, a solid or liquid unit dosage form, a food or a beverage.

### BRIEF DESCRIPTION OF THE DRAWINGS

Exemplary embodiments of the present disclosure are described herein, by way of non-limiting example only, with reference to the following drawings.

FIG. 1. Percent adhesion of AIEC (A) and number of adhering AIEC cells (B) to HT29-MTX cell line, alone and in the presence of Lactobacillus strains SVT01D1 (L. diolivorans), SVT04P1 (L. paracasei), SVT05P2 (L. parafarraginis), SVT06B1 (L. buchneri), SVT07R1 (L. rapi), SVT08Z1 (L. zeae), mean+/−SEM. ^{Ø}p<0.05; ^{#}p≤0.001; *p<0.0001. For SVT01D1, SVT04P1, SVT05P2, SVT06B1, SVT07R1 and SVT08Z1: left hand column, Lactobacilli alone; middle column, Lactobacilli and AIEC co-inoculated; right hand column, Lactobacilli pre-inoculation prior to AIEC.

FIG. 2. Percent adhesion of AIEC (A) and number of adhering AIEC cells (B) to Caco-2 cell line, alone and in the presence of Lactobacillus strains SVT01D1 (L. diolivorans), SVT04P1 (L. paracasei), SVT05P2 (L. parafarraginis), SVT06B1 (L. buchneri), SVT07R1 (L. rapi), SVT08Z1 (L. zeae), mean+/−SEM. ^{Ø}p<0.05; ^{#}p≤0.001; *p<0.0001. For SVT01D1, SVT04P1, SVT05P2, SVT06B1, SVT07R1 and SVT08Z1: left hand column, Lactobacilli alone; middle column, Lactobacilli and AIEC co-inoculated; right hand column, Lactobacilli pre-inoculation prior to AIEC.

FIG. 3. Number of invading AIEC cells to HT29-MTX cell line, alone and in the presence of Lactobacillus strains SVT01D1 (L. diolivorans), SVT04P1 (L. paracasei), SVT05P2 (L. parafarraginis), SVT06B 1 (L. buchneri), SVT07R1 (L. rapi), SVT08Z1 (L. zeae), mean+/−SEM. ^{Ø}p<0.05; ^{#}p≤0.001; *p<0.0001. For SVT01D1, SVT04P1, SVT05P2, SVT06B1, SVT07R1 and SVT08Z1: left hand column, Lactobacilli and AIEC co-inoculated; right hand column, Lactobacilli pre-inoculation prior to AIEC.

FIG. 4. Percent adhesion of HMLN-1 to HT29-MTX and Caco-2 cell lines, alone and in the presence of Lactobacillus strains SVT01D1 (L. diolivorans), SVT04P1 (L. paracasei), SVT05P2 (L. parafarraginis), SVT06B1 (L. buchneri), SVT07R1 (L. rapi), SVT08Z1 (L. zeae), mean+/−SEM. ^{Ø}p<0.05; ^{#}p≤0.001; *p<0.0001. For SVT01D1, SVT04P1, SVT05P2, SVT06B1, SVT07R1 and SVT08Z1: first column, Lactobacilli and HMLN-1 co-inoculated (HT29); second column, Lactobacilli pre-inoculation prior to HMLN-1 infection (HT29); third column, Lactobacilli and HMLN-1 co-inoculated (Caco-2); fourth column, Lactobacilli pre-inoculation prior to HMLN-1 (Caco-2).

FIG. 5. Number of invading HMLN-1 cells into the HT29-MTX and Caco-2 cell lines in the presence of Lactobacillus strains SVT01D1 (L. diolivorans), SVT04P1 (L. paracasei), SVT05P2 (L. parafarraginis), SVT06B1 (L. buchneri), SVT07R1 (L. rapi), SVT08Z1 (L. zeae), mean+/−SEM. ^{Ø}p<0.05; ^{#}p≤0.001; *p<0.0001. For SVT01D1, SVT04P1, SVT05P2, SVT06B1, SVT07R1 and SVT08Z1: first column, Lactobacilli and HMLN-1 co-inoculated (HT29); second column, Lactobacilli pre-inoculation prior to HMLN-1 infection (HT29); third column, Lactobacilli and HMLN-1 co-inoculated (Caco-2); fourth column, Lactobacilli pre-inoculation prior to HMLN-1 infection (Caco-2).

FIG. 6. Number of translocating HMLN-1 cells across the HT29-MTX (A) and Caco-2 (B) cell lines in the presence of Lactobacillus strains SVT01D1 (L. diolivorans), SVT04P1 (L. paracasei), SVT05P2 (L. parafarraginis), SVT06B1 (L. buchneri), SVT07R1 (L. rapi), SVT08Z1 (L. zeae), mean+/−SEM. ^{Ø}p<0.05; ^{#}p≤0.001; *p<0.0001. For SVT01D1, SVT04P1, SVT05P2, SVT06B1, SVT07R1 and SVT08Z1: left hand column, Lactobacilli and HMLN-1 co-inoculated; right hand column, Lactobacilli pre-inoculation prior to HMLN-1 infection.

FIG. 7. Percent adhesion of HMLN-1 and AIEC (A) and number of adhering HMLN-1 cells and AIEC cells (B) to optimised co-culture of HT29-MTX and Caco-2 cell lines, in the presence of Lactobacillus strains SVT01D1 (L. diolivorans), SVT04P1 (L. paracasei), SVT05P2 (L. parafarraginis), SVT06B1 (L. buchneri), SVT07R1 (L. rapi), SVT08Z1 (L. zeae), mean+/−SEM. ^{Ø}p<0.05; ^{#}p≤0.001; *p<0.0001. For SVT01D1, SVT04P1, SVT05P2, SVT06B1, SVT07R1 and SVT08Z1: first column, Lactobacilli and HMLN-1 co-inoculated; second column, Lactobacilli pre-inoculation prior to HMLN-1 infection; third column, Lactobacilli and AIEC co-inoculated; fourth column, Lactobacilli pre-inoculation prior to AIEC infection.

FIG. 8. Number of invading HMLN-1 and AIEC cells into optimised co-culture of HT29-MTX and Caco-2 cell lines in the presence of Lactobacillus strains SVT01D1 (L. diolivorans), SVT04P1 (L. paracasei), SVT05P2 (L. parafarraginis), SVT06B1 (L. buchneri), SVT07R1 (L. rapi), SVT08Z1 (L. zeae), mean+/−SEM. ^{Ø}p<0.05; ^{#}p≤0.001; *p<0.0001. For SVT01D1, SVT04P1, SVT05P2, SVT06B1, SVT07R1 and SVT08Z1: first column, Lactobacilli and HMLN-1 co-inoculated; second column, Lactobacilli pre-inoculation prior to HMLN-1 infection; third column, Lactobacilli and AIEC co-inoculated; fourth column, Lactobacilli pre-inoculation prior to AIEC infection.

FIG. 9. Number of translocating HMLN-1 and AIEC cells across optimised co-culture of HT29-MTX and Caco-2 cell lines in the presence of Lactobacillus strains SVT01D1 (L. diolivorans), SVT04P1 (L. paracasei), SVT05P2 (L. parafarraginis), SVT06B1 (L. buchneri), SVT07R1 (L. rapi), SVT08Z1 (L. zeae), mean+/−SEM. ^{Ø}p<0.05; ^{#}p≤0.001; *p<0.0001. For SVT01D1, SVT04P1, SVT05P2, SVT06B1, SVT07R1 and SVT08Z1: first column, Lactobacilli and HMLN-1 co-inoculated; second column, Lactobacilli pre-inoculation prior to HMLN-1 infection; third column, Lactobacilli and AIEC co-inoculated; fourth column, Lactobacilli pre-inoculation prior to AIEC infection.

FIG. 10. Stool consistency scores in mice of a DSS-induced model of colitis following treatment as described in Example 4. From left to right: Groups 1 to 6, respectively, as described in Example 4. *, p<0.05 Dunnett's test compared to Group 2. ***, p<0.001 Dunnett's test compared to Group 2.

FIG. 11. Faecal blood occurrence scores in mice of a DSS-induced model of colitis following treatment as described in Example 4. From left to right: Groups 1 to 6, respectively, as described in Example 4. *, p<0.05 Dunnett's test compared to Group 2.

FIG. 12. Disease activity index scores in mice of a DSS-induced model of colitis following treatment as described in Example 4. From left to right: Groups 1 to 6, respectively, as described in Example 4. *, p<0.05 Dunnett's test compared to Group 2.

FIG. 13. Comparative efficacy of drug therapies for chronic DSS-induced ulcerative colitis in a murine model as measured by disease activity index (DAI) scores over 29 days.

FIG. 14. Faecal blood occurrence (A), stool consistency scores (B) and disease activity index (DAI) scores (C) in mice of a DSS-induced model of colitis following treatment as described in Example 4A. From left to right: Groups 1, 2, 7 and 6 as described in Example 4A. *, p<0.05 Dunnett's test compared to Group 2; **, p<0.01 Dunnett's test compared to Group 2; ***, p<0.001 Dunnett's test compared to Group 2.

FIG. 15. Cytokine expression analysis (A, IL-6; B, TNFα) in mice of a DSS-induced model of colitis following treatment as described in Example 4A. From left to right: Groups 1, 2, 7 and 6 as described in Example 4A. *, p<0.05 Dunnett's test compared to Group 2.

FIG. 16. Total, proximate, middle and distal composite scores for ulceration and inflammation in mice of a DSS-induced model of colitis following treatment as described in Example 4A. From left to right: Groups 1, 2, 7 and 6 as described in Example 4A. *, p<0.05 Dunnett's test compared to Group 2; **, p<0.01 Dunnett's test compared to Group 2; ***, p<0.001 Dunnett's test compared to Group 2.

### DETAILED DESCRIPTION

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

As used herein the term “effective amount” includes within its meaning a non-toxic but sufficient amount of composition to provide the desired therapeutic effect. The exact amount required will vary from subject to subject depending on factors such as the species being treated, the age and general condition of the subject, the severity of the condition being treated, the particular agent being administered and the mode of administration and so forth. For any given case, an appropriate “effective amount” may be determined by one of ordinary skill in the art using only routine experimentation.

The term “subject” as used herein refers to mammals and includes humans, primates, livestock animals (e.g. cattle, dairy cows, horses, sheep, pigs), laboratory test animals (e.g. mice, rabbits, rats, guinea pigs), companion animals (e.g. dogs, cats), performance animals (e.g. racehorses), and captive wild animals. In exemplary embodiments, the mammal is human.

As used herein the terms “treating”, “treatment” and the like refer to any and all applications which remedy, or otherwise hinder, retard, or reverse the progression of, inflammation, an infection or of a condition, or at least one symptom of such inflammation, infection or condition, including reducing the severity of the inflammation, infection or condition. Thus, treatment does not necessarily imply that a subject is treated until complete elimination of, or recovery from, the inflammation, infection or condition. Similarly, the terms “preventing”, “prevention” and the like refer to any and all applications which prevent the establishment of condition or otherwise delay the onset of such inflammation, infection or condition.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

In the context of this specification, the term “microbial biotherapeutic” is to be given its broadest construction and is understood to refer to a microbial cell population or preparation, or component of a microbial cell population or preparation, which when administered to a subject in an effective amount promotes a health benefit in the subject.

In the context of this specification, the term “prebiotic” is to be given its broadest construction and is understood to refer to any non-digestible substance that stimulates the growth and/or activity of commensal beneficial bacteria in the digestive system.

In the context of this specification, the terms “food”, “foods”, “beverage” or “beverages” include but are not limited to health foods and beverages, functional foods and beverages, and foods and beverages for specified health use. When such foods or beverages of the present invention are used for subjects other than humans, the terms can be used to include a feedstuff.

Provided herein are methods for treating or preventing inflammation, or an inflammatory or autoimmune condition, or one or more symptoms associated therewith, comprising administering to a subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

As used herein, the term “inflammatory condition” typically refers to a condition characterised by inflammation, or the complex biological response to a noxious stimulus such as infection by a microbial pathogen and/or virus. The clinical features of an inflammatory condition is likely to depend on the noxious stimulus (or stimuli), but may be characterised by heat, pain, redness or swelling of the affected organ or tissue. The inflammatory condition may be acute or chronic.

Also provided herein are methods for treating or preventing a condition of the gastrointestinal tract, urinary tract, skin, nails or joints, comprising administering to a subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured, wherein the conditions is associated with inflammation of the gastrointestinal tract, urinary tract, skin, nails or joints and/or wherein the condition is caused by or associated with an infection of the gastrointestinal tract, urinary tract, skin, nails or joints.

In the following discussion, in the context of administration of the Lactobacillus species or culture supernatants or cell free filtrates derived from culture media in which the Lactobacillus has been cultured, and in the context of compositions comprising the same, the term “Lactobacillus” may be used to refer not only to the specific Lactobacillus species defined herein per se, but also more broadly to refer to culture supernatants or cell free filtrates derived from culture media in which the specific Lactobacillus species defined herein have been cultured.

In particular embodiments the inflammation may be inflammation of the gastrointestinal tract, urinary tract, skin, nails or joints. The inflammation may be caused or induced by, or otherwise associated with, an infection, for example a bacterial, viral or parasitic infection. In exemplary embodiments the pathogen causing the infection is a bacteria. Exemplary bacteria responsible for such infections are described herein below.

The gastrointestinal inflammation may be associated with one or more conditions affecting the gastrointestinal tract, which conditions may be characterised by or may lead to inflammation, for example food poisoning, diarrhoea, ulcers such as gastric ulcers and mouth ulcers, dental caries and periodontal disease. The inflammation may be an acute episode or may be chronic inflammation.

The one or more symptoms associated with gastrointestinal inflammation may include, for example, diarrhoea, poor stool consistency, faecal blood presence, abdominal cramping, abdominal bloating, abdominal pain, ulceration of a gastrointestinal epithelial lining such as of the mouth, stomach or small or large intestine, or swelling of the gums. Those skilled in the art will readily appreciate that the scope of the present disclosure should not be limited by reference these exemplary symptoms, and there are other symptoms of inflammation of the gastrointestinal tract that will be encompassed by the present disclosure.

The condition may be an acute condition or a chronic condition. Examples of inflammatory and autoimmune conditions and related conditions affecting the gastrointestinal tract to which the present disclosure relates include, but are not limited to, inflammatory bowel diseases, irritable bowel syndrome, gastritis, gastroenteritis, gingivitis, pharyngitis (such as streptococcal pharyngitis or Strep throat), ileitis, and other conditions caused by bacterial infections such as C. difficile gastritis and yersiniosis. In exemplary embodiments the condition is an inflammatory bowel disease. The inflammatory bowel disease may be colitis, such as, for example, ulcerative colitis, Crohn's disease, ischemic colitis, enterocolitis or antibiotic-associated hemorrhagic colitis (AAHC). AAHC may be caused by a Klebsiella species such as Klebsiella oxytoca. The ulcerative colitis may be acute or chronic ulcerative colitis. In an exemplary embodiment the ulcerative colitis is chronic ulcerative colitis.

Embodiments of the present disclosure provide methods for treating or preventing at least one symptom of a gastrointestinal infection, such as a bacterial infection (e.g Salmonella, E. coli, Listeria, B. cereus), viral infection (e.g. norovirus, rotavirus) or parasitic infection (e.g. Giardia, Cryptosporidium, Ascaris, Eimeria or Trichinella). The at least one symptom may be poor stool consistency, diarrhoea, faecal blood, abdominal cramping, abdominal bloating or abdominal pain. Thus, methods of the present disclosure may prove effective, for example, for travellers, as a preventative or treatment for, or to reduce the severity of food poisoning. The at least one symptom may be associated with, for example, irritable bowel syndrome.

Conditions and infections of the mouth and throat that may be treated in accordance with the present disclosure include, for example, pharyngitis such as Streptococcal pharyngitis, tonsillitis, halitosis and scarlet fever.

The inflammation or condition of the urinary tract may be, for example, a condition of the kidneys, ureters, bladder or urethra. Exemplary conditions include, but are not limited to, urinary tract infections and associated conditions such as cystitis, urethritis, pyelonephritis, renal abscesses, and asymptomatic bacteriuria. The urinary tract infection or associated condition be associated with a drainage device such as a urinary catheter.

The method may be employed to treat one or more symptoms associated with a urinary tract infection or associated condition. Such symptoms include, but are not limited to, dysuria (painful urination), urgency, hesitancy, frequency of urination, polyuria, incomplete voiding, hematuria, urinary incontinence, cloudy urine or a burning sensation when urinating. Those skilled in the art will readily appreciate that the scope of the present disclosure should not be limited by reference these exemplary symptoms, and there are other symptoms of urinary tract infections that will be encompassed by the present disclosure.

The inflammation or condition of the skin or nails may be, for example, psoriasis, dermatitis, eczema, rosacea, acne, ichtyosis, fungal skin and/or nail infection or other skin condition characterized by or associated with inflammation, plaques or skin lesions. Exemplary forms of psoriasis include plaque psoriasis, guttate psoriasis and pustular psoriasis. Exemplary forms of dermatitis include atopic dermatitis, infant dermatitis, seborrhic dermatitis, contact dermatitis, occupational dermatitis, hand dermatitis, nummular dermatitis, stasis dermatitis, perioral dermatitis and dermatitis herpetiformis. Exemplary fungal infections include tinea pedis (Athlete's foot), tinea cruris (tinea of the groin, Jock itch), tinea capitis (tinea of the head and scalp), tinea corporis (tinea of the body) and tinea unguium (tinea of a fingernail or toenail, onychomycosis). The inflammation or inflammatory condition of the skin or nails, may be caused by, or be associated with, a bacterial, fungal or viral infection.

The inflammatory joint condition may be arthritis. The arthritis may be, for example, rheumatoid arthritis or osteoarthritis.

Embodiments of the present disclosure also provide methods for inhibiting or reducing inflammation or one more symptoms associated with inflammation, in particular of the gastrointestinal tract, urinary tract, skin, nails or joints. The term “inhibiting” and variations thereof such as “inhibition”, “inhibits”, “reduces”, “reducing” and the like, are used interchangeably herein to denote an improvement (i.e., reduction) in the severity of inflammation, or in the severity of the condition, or in the severity of infection, or in at least one symptom of the inflammation, condition or infection.

Other exemplary inflammatory or autoimmune conditions include, for example, disorders such as rheumatic fever, chronic fatigue syndrome, systemic lupus erythematosus, Sjogren's syndrome, inflammation of the prostate, pelvic inflammatory disease, pancreatitis, vasculitis, inflammation of the feet including gout, and period pain.

Methods of the present disclosure also relate to the treatment or prevention of bacterial infections of the gastrointestinal tract, urinary tract, skin, nails or joints. The methods comprise administering to a subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

Methods of the present disclosure also relate to the treatment or prevention of bacterial infections that cause, induce or are otherwise associated with inflammation, or an inflammatory or autoimmune condition of the gastrointestinal tract, urinary tract, skin, nails, or joints such as those described herein. In the present context “induce” means stimulating or contributing to the development or exacerbation of inflammation or an inflammatory or autoimmune condition, optionally in concert with one or more other factors. The methods comprise administering to a subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

Bacterial infections to which embodiments of the disclosure relate may be caused by pathogenic Gram-negative or Gram-positive bacteria. Exemplary pathogenic bacteria include, but are not limited to, members of the Enterobacteriaceae, such as, for example, Escherichia coli, Yersinia species, Enterobacter species, Salmonella species, Shigella species, Klebsiella species, Proteus species and Citrobacter species. Exemplary Enterobacteriaceae include adherent-invasive E. coli (AIEC strains), enteropathogenic E. coli (EPEC strains), enterotoxigenic E. coli (ETEC strains), enterohemorrhagic E. coli (EHEC strains), uropathogenic E. coli (UPEC strains), Yersinia enterolitica, Enterobacter cloacae, Salmonella typhimurium, Salmonella enterica, Salmonella enteridis, Shigella flexneri, Shigella boydii, Shigella sonnei, Shigella dysenteriae, Klebsiella oxytoca and Proteus mirabilis. Other exemplary bacterial pathogens against which methods of the present disclosure may be employed include Helicobacter species such as Helicobacter pylori, Campylobacter species such as Campylobacter jejuni, Pseudomonas species such as Pseudomonas aeruginosa, Vibrio species such as Vibrio cholerae, a Clostridium species such as Clostridium difficile, Streptococcus species such as Streptococcus mutans, Streptococcus pyogenes and other Group A (haemolytic) Streptococcus, and Group B Streptococcus, Staphylococcus species such as Staphylococcus aureus and Staphylococcus saprophyticus, Enterococcus species such as Enterococcus faecalis and Enterococcus faecium, and Mycobacterium species such as Mycobacterium avium subspecies paratuberculosis.

Gastrointestinal infections that may be treated in accordance with the present disclosure may be caused by, for example, one or more of Escherichia coli, an Escherichia coli AIEC strain, Yersinia enterolitica, Salmonella typhimurium, Salmonella enteridis, Shigella flexneri, Shigella boydii, Shigella sonnei, Shigella dysenteriae, Klebsiella oxytoca, Proteus mirabilis, Helicobacter pylori, Pseudomonas aeruginosa, Campylobacter jejuni, Vibrio cholera, Clostridium difficile, Streptococcus mutans, Streptococcus pyogenes, Group A (haemolytic) Streptococcus, Staphylococcus aureus and Mycobacterium avium subspecies paratuberculosis. Infections of the moth or throat that may be treated in accordance with the present disclosure may be caused by, for example, one or more of Streptococcus mutans, Streptococcus pyogenes, and other Group A (haemolytic) Streptococcus. Urinary tract infections that may be treated in accordance with the present disclosure may be caused by, for example, one or more of Escherichia coli UPEC strains, Enterococcus faecalis, Enterococcus faecium, Klebsiella oxytoca, Proteus mirabilis, Pseudomonas aeruginosa, Group B Streptococcus, Staphylococcus aureus and Staphylococcus saprophyticus.

Also provided herein are methods for inhibiting or preventing adhesion of a bacterial pathogen to the gastrointestinal mucosa of a subject and for inhibiting or preventing invasion of gastrointestinal epithelial cells of a subject by a bacterial pathogen. Said methods comprise administering to the subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

In this context, as used herein “inhibit”, “inhibiting” and the like refer to a reduction in adhesion and/or invasion of gastrointestinal epithelial lining or cells by a bacterial pathogen in the presence of one or more of the Lactobacillus species defined herein compared to that which would occur in the absence of the Lactobacillus species.

The bacterial pathogen may be capable of adhering to the mucosal epithelial lining of any part of the gastrointestinal tract and/or be capable of invading epithelial cells lining any part of the gastrointestinal tract. In exemplary embodiments, the bacterial pathogen may be a member of the Enterobacteriaceae, such as, for example, Escherichia coli, Yersinia enterolitica, a Salmonella species or a Shigella species. The E coli may be, for example, an adherent-invasive E. coli (AIEC), an enteropathogenic E. coli (EPEC), an enterotoxigenic E. coli (ETEC), or an enterohemorrhagic E. coli (EHEC). The Salmonella species may be, for example, Salmonella typhimurium, Salmonella enterica or Salmonella enteritidis. The Shigella species may be, for example, Shigella flexneri, Shigella boydii, Shigella sonnei or Shigella dysenteriae.

Methods of the present disclosure also relate to the treatment or prevention of bacterial infections of the gastrointestinal tract, for example infections that cause, induce or may otherwise be associated with inflammation and inflammatory and autoimmune conditions of the gastrointestinal tract such as those exemplified hereinbefore. In the present context “induce” means stimulating or contributing to the development or exacerbation of inflammation or an inflammatory or autoimmune condition, optionally in concert with one or more other factors. The methods comprise administering to a subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

Also provided herein are methods for promoting wound healing, comprising administering a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured

As used herein in the context of wound healing, the terms “promoting”, “promotion” and variations thereof refer to the ability of a combination or composition disclosed herein to induce, enhance or otherwise advance the natural processes associated with wound healing and/or tissue regeneration associated therewith. In embodiments the promotion may be relative to the healing observed in the absence of administration of the combination or composition. The promotion may be direct or indirect. It will be understood that in indirectly promoting wound healing, the combination or composition may affect the expression or activity of molecules which themselves regulate or otherwise influence, either directly or indirectly, the wound healing or tissue regeneration processes. The promotion may be qualitative, quantitative and/or temporal. That is, for example, the administration of the combination or composition may result in more rapid wound healing and/or tissue regeneration than would occur in the absence of such administration.

The wound may be, for example, a surgical wound, incision or superficial wound such as a cut, graze, contusion or bruise. The wound may be a chronic wound such as a pressure sore, pressure ulcer, diabetic foot ulcer or severe burn.

Methods of the present disclosure employ the administration of one or more Lactobacillus species selected from L. parafarraginis, L. buchneri, L. zeae, L. rapi, L. paracasei, and L. diolivorans and compositions comprising one or more of these species. In view of some taxonomic discrepancies and uncertainties, L. zeae may also be referred to elsewhere as L. casei. However for the purposes of the present disclosure the L. zeae nomenclature is retained.

Methods of the present disclosure may comprise the administration of two, three, four, five or all six of the Lactobacillus species Lactobacillus buchneri, Lactobacillus zeae, Lactobacillus rapi, Lactobacillus paracasei, Lactobacillus parafarraginis, and Lactobacillus diolivorans, or culture supernatants or cell free filtrates derived from culture media in which two, three, four, five or all six of said Lactobacillus have been cultured. In such embodiments the bacteria may be cultured together or separately.

The Lactobacillus parafarraginis may be Lactobacillus parafarraginis Lp18 available under Accession Number V11/022945, previously described in WO2013/063658. The L. parafarraginis may be L. parafarraginis SVT-18 (which may be elsewhere referred to by the alternate designation SVT-05P2) deposited pursuant to the Budapest Treaty with the Belgium Co-Ordinated Collection of Micro-organisms (BCCM) on 27 Feb. 2019 under Accession Number LMG P-31292.

The Lactobacillus buchneri may be Lactobacillus buchneri Lb23 available under Accession Number V11/022946, previously described in WO2013/063658. The L. buchneri may be L. buchneri SVT-23 (which may be elsewhere referred to by the alternate designation SVT-06B1) deposited pursuant to the Budapest Treaty with the Belgium Co-Ordinated Collection of Micro-organisms (BCCM) on 27 Feb. 2019 under Accession Number LMG P-31293.

The Lactobacillus zeae may be Lactobacillus zeae Lz26 available under Accession Number V11/022948, previously described in WO2013/063658. The L. zeae may be L. zeae SVT-26 (which may be elsewhere referred to by the alternate designation SVT-08Z1) deposited pursuant to the Budapest Treaty with the Belgium Co-Ordinated Collection of Micro-organisms (BCCM) on 27 Feb. 2019 under Accession Number LMG P-31295.

The L. rapi may be L. rapi Lr24 available under Accession Number V11/022947, previously described in WO2013/063658. The L. rapi may be L. rapi SVT-24 (which may be elsewhere referred to by the alternate designation SVT-07R1) deposited pursuant to the Budapest Treaty with the Belgium Co-Ordinated Collection of Micro-organisms (BCCM) on 27 Feb. 2019 under Accession Number LMG P-31294.

The Lactobacillus paracasei may be Lactobacillus paracasei Lp9 available under Accession Number V12/022849, previously described in WO2014/172758 (designated as strain ‘T9’ therein). The L. paracasei may be L. paracasei SVT-09 (which may be elsewhere referred to by the alternate designation SVT-04P1) deposited pursuant to the Budapest Treaty with the Belgium Co-Ordinated Collection of Micro-organisms (BCCM) on 27 Feb. 2019 under Accession Number LMG P-31290.

The Lactobacillus diolivorans may be Lactobacillus diolivorans Ld3 available under Accession Number V12/022847, previously described in WO2014/172758 (designated as strain ‘N3’ therein). The L. diolivorans may be L. diolivorans SVT-03 (which may be elsewhere referred to by the alternate designation SVT-01D1) deposited pursuant to the Budapest Treaty with the Belgium Co-Ordinated Collection of Micro-organisms (BCCM) on 27 Feb. 2019 under Accession Number LMG P-31287.

The concentrations of individual Lactobacillus species to be administered in accordance with methods of the present disclosure will depend on a variety of factors including the identity and number of individual species employed, the exact nature and severity of the inflammation, condition or infection to be treated or prevented, the form in which a composition is applied and the means by which it is applied. For any given case, appropriate concentrations may be determined by one of ordinary skill in the art using only routine experimentation. By way of example only, the concentration of the Lactobacillus species, or each species present in the case of a combination, may be from about 1×10² cfu/ml to about 1×10¹¹ cfu/ml, and may be about 1×10³ cfu/ml, about 2.5×10³ cfu/ml, about 5×10³ cfu/ml, 1×10⁴ cfu/ml, about 2.5×10⁴ cfu/ml, about 5×10⁴ cfu/ml, 1×10⁵ cfu/ml, about 2.5×10⁵ cfu/ml, about 5×10⁵ cfu/ml, 1×10⁶ cfu/ml, about 2.5×10⁶ cfu/ml, about 5×10⁶ cfu/ml, 1×10⁷ cfu/ml, about 2.5×10⁷ cfu/ml, about 5×10⁷ cfu/ml, 1×10⁸ cfu/ml, about 2.5×10⁸ cfu/ml, about 5×10⁸ cfu/ml, 1×10⁹ cfu/ml, about 2.5×10⁹ cfu/ml, or about 5×10⁹ cfu/ml, about 1×10¹⁰ cfu/ml, about 1.5×10¹⁰ cfu/ml, about 2.5×10¹⁰ cfu/ml, about 5×10¹⁰ cfu/ml or about 1×10¹¹ cfu/ml.

Also contemplated by the present disclosure is the use of variants of the Lactobacillus species described herein. As used herein, the term “variant” refers to both naturally occurring and specifically developed variants or mutants of the species disclosed and exemplified herein. Variants may or may not have the same identifying biological characteristics of the specific species exemplified herein, provided they share similar advantageous properties in terms of treating or preventing inflammatory conditions. Illustrative examples of suitable methods for preparing variants exemplified herein include, but are not limited to, gene integration techniques such as those mediated by insertional elements or transposons or by homologous recombination, other recombinant DNA techniques for modifying, inserting, deleting, activating or silencing genes, intraspecific protoplast fusion, mutagenesis by irradiation with ultraviolet light or X-rays, or by treatment with a chemical mutagen such as nitrosoguanidine, methylmethane sulfonate, nitrogen mustard and the like, and bacteriophage-mediated transduction. Suitable and applicable methods are well known in the art and are described, for example, in J. H. Miller, Experiments in Molecular Genetics, Cold Spring Harbor Laboratory Press, Cold Spring Harbor, N.Y. (1972); J. H. Miller, A Short Course in Bacterial Genetics, Cold Spring Harbor Laboratory Press, Cold Spring Harbor, N.Y. (1992); and J. Sambrook, D. Russell, Molecular Cloning: A Laboratory Manual, 3rd ed., Cold Spring Harbor Laboratory Press, Cold Spring Harbor, N.Y. (2001), inter alia.

Also encompassed by the term “variant” as used herein are microbial strains phylogenetically closely related to species disclosed herein and strains possessing substantial sequence identity with the species disclosed herein at one or more phylogenetically informative markers such as rRNA genes, elongation and initiation factor genes, RNA polymerase subunit genes, DNA gyrase genes, heat shock protein genes and recA genes. For example, the 16S rRNA genes of a “variant” strain as contemplated herein may share about 85%, 86%, 87%, 88%, 89%, 90%, 91%, 92%, 93%, 94%, 95%, 96%, 97%, 98% or 99% sequence identity with a strain disclosed herein.

The Lactobacillus species described herein, and combinations thereof, or culture supernatants or cell free filtrates derived from culture media are typically administered in accordance with the present disclosure in the form of a composition. In embodiments in which combinations of species, or culture supernatants or cell free filtrates derived from culturing multiple species, those skilled in the art will appreciate that each of the species, supernatants or filtrates to be administered need not be contained in the same composition. Where administration is separate, administration may be sequential or simultaneous.

Compositions for use in accordance with the present disclosure may be prepared by admixing the relevant components and formulating the resulting mixture into a dosage form that is suitable for administration to a subject. Accordingly, the compositions may comprise pharmaceutically acceptable carriers, diluents, excipients and/or adjuvants. The carriers, diluents, excipients and adjuvants must be “acceptable” in terms of being compatible with other components of the composition, and not deleterious to the subject who is to receive the composition. Methods for preparing suitable compositions for administration, and carriers, diluents, excipients and adjuvants suitable for use in compositions formulated for topical, oral or sublingual administration are well known to those skilled in the art. In exemplary embodiments, the composition is formulated with a carrier comprising sterile isotonic saline or 3% sucrose.

Compositions may be administered via any convenient or suitable route, variety of routes including, but not limited to, oral, sublingual, buccal, rectal, topical, intranasal, intraocular, transmucosal, intestinal, enteral, intramuscular, subcutaneous, intramedullary, intrathecal, intraventricular, intracerebral, intravesical, intravenous or intraperitoneal. The appropriate route may depend, for example, on the nature and severity of the inflammation, condition or infection to be treated or prevented and the site of the inflammation, condition or infection. The compositions may be administered in any suitable form, typically in solid or liquid form. For example, the compositions may be formulated using methods and techniques well known to those skilled in the art, into tablets, troches, capsules, caplets, elixirs, suspensions, syrups, wafers, granules, powders, gels, pastes, solutions, creams, sprays, suspensions, soluble sachets, lozenges, effervescent tablets, chewable tablets, multi-layer tablets, and the like. For oral administration, the Lactobacillus or compositions may be conveniently incorporated in a variety of beverages, food products, nutraceutical products, nutritional supplements, food additives, pharmaceuticals, over-the-counter formulations and animal feed supplements. For topical application, suitable vehicles include, but are not limited to, lotions, liniments, gels, creams, ointments, foams, sprays, oils, powders and the like. Compositions may also be impregnated into transdermal patches, plasters, and wound dressings such as bandages or hydrocolloid dressings, typically in liquid or semi-liquid form.

As will be appreciated by those skilled in the art, the choice of pharmaceutically acceptable carrier or diluent will be dependent on the route of administration and on the nature and severity of the condition and the subject to be treated. The particular carrier or delivery system and route of administration may be readily determined by a person skilled in the art. A person skilled in the art will readily be able to determine appropriate formulations useful in the methods of the disclosure using conventional approaches.

For example, compositions of the present disclosure may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives and hydrophilic beeswax derivatives.

Alternatively, the Lactobacillus can be formulated readily using pharmaceutically acceptable carriers well known in the art into dosages suitable for oral administration. These carriers may be selected from sugars, starches, cellulose and its derivatives, malt, gelatine, talc, calcium sulfate, vegetable oils, synthetic oils, polyols, alginic acid, phosphate buffered solutions, emulsifiers, isotonic saline and pyrogen-free water.

Some examples of suitable carriers, diluents, excipients and adjuvants for oral use include liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration. Adjuvants typically include emollients, emulsifiers, thickening agents, preservatives, bactericides and buffering agents. For administration as an injectable solution or suspension, non-toxic parenterally acceptable diluents or carriers can include, Ringer's solution, isotonic saline, phosphate buffered saline, ethanol and 1,2 propylene glycol.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof. Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like. emulsions for oral administration may further comprise one or more emulsifying agents. Suitable emulsifying agents include dispersing agents as exemplified above or natural gums such as guar gum, gum acacia or gum tragacanth.

Methods for preparing suitable parenterally administrable compositions will be well known to those skilled in the art, and are described in more detail in, for example, Remington's Pharmaceutical Science, 15th ed., Mack Publishing Company, Easton, Pa., hereby incorporated by reference herein.

For compositions formulated for topical administration, examples of pharmaceutically acceptable diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenylpolysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly.

In further embodiments, the composition may further comprise suspending agents and/or humectants, such as povidone or propylene glycol, and neutralising agents for adjusting the viscosity of the composition, such as sodium hydroxide, triethanolamine (TEA) or ethylenediamine tetraacetic acid (EDTA).

Compositions of the present disclosure may be administered, for example one or more times a week, optionally for example once a week, once every second day, once a day, twice a day or three times a day, depending on the condition to be treated or prevented, the severity of the condition and the desired outcome. The duration of administration by a subject will also vary depending on the condition to be treated or prevented, the severity of the condition and the desired outcome. The amount of composition to be administered by a subject will vary depending on a range of factors including the identity of the microorganisms administered, the nature and severity of the condition to be treated or prevented, the age and general wellbeing of the subject, and the desired outcome. Suitable dosage regimes can readily be determined by the skilled addressee.

In exemplary embodiments, an about 1 ml to about 25 ml liquid formulation of a Lactobacillus species at a final concentration of between about 10⁵ and 10¹¹ cfu/ml may be administered to a subject on a once-a-day, twice-a-day or more frequent basis. The volume of the liquid formulation may be, for example, about 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, or 25 ml.

The Lactobacillus may be combined with other therapeutic agents for example, but not limited to, antibiotics, antimicrobial agents, antiseptics, anaesthetics, anti-infective agents, anti-inflammatory agents, immunosuppressive agents and other therapeutic agents indicated for the treatment of inflammatory conditions such as steroids, and NSAIDs. Administration of such additional agents may be at the same time or at different times, i.e. simultaneous or sequential, and may be administered by the same or different routes, with respect to compositions comprising the microorganisms the subject of the present disclosure. Additional therapeutic agents may be co-formulated with microorganisms used in the methods.

Non-limiting examples of additional anti-inflammatory agents that may be employed include steroidal and non-steroidal compounds such as clobetasol propionate, betamethasone dipropionate, halobetasol proprionate, diflorasone diacetate, fluocinonide, halcinonide, amcinonide, desoximetasone, triamcinolone acetonide, mometasone furoate, fluticasone propionate, betamethasone dipropionate, fluocinolone acetonide, hydrocortisone valerate, hydrocortisone butyrate, flurandrenolide, triamcinolone acetonide, mometasone furoate, triamcinolone acetonide, fluticasone propionate, desonide, fluocinolone acetonide, hydrocortisone valerate, prednicarbate, triamcinolone acetonide, desonide, hydrocorti-sone, hydrocortisone aceponate, hydrocortisone buteprate, methylprednisolone aceponate, mometasone furoate and prednicarbate. Non-limiting examples of suitable non-steroidal anti-inflammatory compounds include indomethacin, ketoprofen, felbinac, diclofenac, ibuprofen, piroxicam, benzydamin, acetylsalicylic acid, diflunisal, salsalate, naproxen, fenoprofen, ketoprofen, flurbiprofen, oxaprozin, loxoprofen, indomethacin, sulindac, etodolac, ketorolac, diclo-fenac, nabumetone, piroxicam, meloxicam, tenoxicam, droxicam, lornoxicam, isoxicam, mefenamic acid, meclofenamic acid, flufenamic acid, tolfenamic acid, firocoxib, and licofelone, semi-synthetic glycosaminoglycosan ethers, flavanols, flavonoids, isoflavones and derivatives. The anti-inflammatory agent may be a suppressor of cytokine signalling such as, for example, cyclosporin A, 6-thioguanine, sulfasalazine, mesalamine (5-aminosalicylic acid), etanercept, prednisolone, or balsalazide.

The anti-infective agent may be any agent which treats an infection in a subject. In particular embodiments, the anti-infective agent is able to kill or inhibit the growth of an infectious organism which is capable of being transferred, in entirety or in part, between cells via an apoptotic body. Suitable anti-infective agents include, but are not limited to, an anti-viral agent, an anti-bacterial agent, an anti-protozoal agent, an anti-fungal agent or a combination thereof.

Illustrative anti-viral agents include, but are not limited to, abacavir sulfate, acyclovir especially acyclovir sodium, adefovir, amantadine especially amantadine hydrochloride, amprenavir, ampligen, atazanavir, cidofovir, darunavir, delavirdine especially delavirdine mesylate, didanosine, docosanol, dolutegravir, edoxudine, efavirenz, emtricitabine, elvitegravir, enfuvirtide, entecavir, famciclovir, fomivirisen especially fomivirsen sodium, foscarnet especially foscarnet sodium, ganciclovir, ibacitabine, idoxuridine, imiquimod, indinavir especially indinavir sulfate, inosine pranobex, lamivudine, lopinavir, maraviroc, metisazone, moroxydine, nelfinavir especially nelfinavir mesylate, nevirapine, nitazoxanide, oseltamivir particularly oseltamivir phosphate, penciclovir, peramivir, pleconaril, podophyllotoxin, raltegravir, ribavirin, rimantadine especially rimantadine hydrochloride, ritonavir, saquinavir especially saquinavir mesylate, sofosbuvir, stavudine, telaprivir, tenofovir, tipranovir, trifluridine, tromantadine, umifenovir, valacyclovir especially valacyclovir hydrochloride, valganciclovir, vicriviroc, vidarabine, viramidine, zalcitabine, zanamivir, zidovudine and pharmaceutically acceptable salts and combinations thereof.

Illustrative anti-bacterial agents include, but are not limited to, quinolones (e.g. amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, clinafloxacin, gatifloxacin, moxifloxacin, gemifloxacin, and garenoxacin), tetracyclines, glycylcyclines and oxazolidinones (e.g. chlortetracycline, demeclocycline, doxycycline, lymecycline, methacycline, minocycline, oxytetracycline, tetracycline, tigecycline; linezolide, eperezolid), glycopeptides, aminoglycosides (e.g. amikacin, arbekacin, butirosin, dibekacin, fortimicins, gentamicin, kanamycin, menomycin, netilmicin, ribostamycin, sisomicin, spectinomycin, streptomycin, tobramycin), β-lactams (e.g. imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephacetrile, cephalexin, cephaloglycin, cephaloridine, cephalothin, cephapirin, cephradine, cefinetazole, cefoxitin, cefotetan, azthreonam, carumonam, flomoxef, moxalactam, amdinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, LY206763), rifamycins, macrolides (e.g. azithromycin, clarithromycin, erythromycin, oleandomycin, rokitamycin, rosaramicin, roxithromycin, troleandomycin), ketolides (e.g. telithromycin, cethromycin), coumermycins, lincosamides (e.g. clindamycin, lincomycin), chloramphenicol, clofazimine, cycloserine, dapsone, ethambutol hydrochloride, isoniazid, pyrazinamide, rifabutin, rifampin, rifapentine and streptomycin sulfate.

Illustrative anti-protozoal agents include, but are not limited to, atovaquone, metronidazole including metronidazole hydrochloride, pentamidine including pentamidine isethionate, chloroquine including chloroquine hydrochloride and chloroquine phosphate, doxycycline, hydroxychloroquine sulfate, mefloquine including mefloquine hydrochloride, primaquine including primaquine phosphate, pyrimethamine, pyrimethamine with sulfadoxine, trimethoprim, sulfamethoxazole, clindamycin, quinine, quinidine, sulfadiazine, artemether, lumefantrine, artesunate, nitazoxanide, suramin, melarsoprol, eflornithine, nifurtimox, stibogluconate including sodium stibogluconate, amphotericin B including liposomal amphotericin B, miltefosine, paromomycin, ketoconazole, itraconazole, fluconazole, and pharmaceutically acceptable salts and combinations thereof.

Illustrative anti-fungal agents include, but are not limited to, abafungin, albaconazole, amorolfine, amphotericin B, amphotericin B cholesteryl sulfate complex, amphotericin B lipid complex, amphotericin B liposomal, anidulafungin, bifonazole, butenafine, butoconazole, candicidin, caspofungin, clotrimazole, econazole, efinaconazole, fenticonazole, fluconazole, flucytosine, griseofulvin microsize, griseofulvin ultramicrosize, hamycin, isavuconazole, isoconazole, itraconazole, ketoconazole, luliconazole, micafungin, miconazole, naftifine, natamycin, nystatin, omoconazole, oxiconazole, posaconazole, propiconazole, ravuconazole, sertaconazole, sulconazole, terbinafine including terbinafine hydrochloride, terconazole, tioconazole, voriconazole, and pharmaceutically acceptable salts and combinations thereof.

Illustrative immunosuppressive agents include, but are not limited to: corticosteroids such as, for example, budesonide, prednisone and prednisolone; mTOR inhibitors such as, for example, sirolimus and everolimus; and monoclonal antibodies such as, for example, adalimumab, infliximab, certolizumab, natalizumab, ustekinumab and vedolizumab, and biosimilars thereof.

In exemplary embodiments the Lactobacillus described herein are provided and administered in the form of microbial biotherapeutic compositions. Such compositions may further comprise one or more additional microorganisms such as, for example, Lactobacillus rhamnosus, Lactobacillus plantarum, Lactobacillus bulgaricus, Lactobacillus casei, Lactobacillus acidophilus, Lactobacillus fermentum, Lactococcus lactis, Streptococcus thermophilus, Bifidobacterium breve, Bifidobacterium bifidum, Bifidobacterium lactis, and Bifidobacterium animalis.

Microbial biotherapeutic compositions may comprise one or more prebiotic components. Suitable prebiotics include, for example, polydextrose, inulin, fructooligosaccharides (FOS), xylooligosaccharides (XOS), galactooligosaccharides (GOS), mannan oligosaccharides, protein-based green lipped mussel extract, and various prebiotic-containing foods such as raw onion, raw leek, raw chickory root and raw artichoke. In certain embodiments the prebiotic is a fructooligosaccharide.

Compositions comprising Lactobacillus as described herein may be administered in any suitable form, including any of the dosage forms described above. The microbial biotherapeutic compositions may be provided to the user in a powder form, suitable for mixing by the user into any type of drink or food product (for example water, fruit juice or yoghurt) or for consumption as a powder in the absence of a drink or additional food product. The microbial biotherapeutic compositions may therefore be conveniently incorporated in a variety of food and/or beverage products, nutraceutical products, supplements, food additives, and over-the-counter formulations. The food or food additive may be a solid form such as a powder, or a liquid form. Specific examples of the types of beverages or foods include, but are not limited to water-based, milk-based, yoghurt-based, other dairy-based, milk-substitute based such as soy milk or oat milk, or juice-based beverages, water, soft drinks, carbonated drinks, and nutritional beverages, (including a concentrated stock solution of a beverage and a dry powder for preparation of such a beverage); baked products such as crackers, breads, muffins, rolls, bagels, biscuits, cereals, bars such as muesli bars, health food bars and the like, dressings, sauces, custards, yoghurts, puddings, pre-packaged frozen meals, soups and confectioneries.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present disclosure will now be described with reference to the following specific examples, which should not be construed as in any way limiting the scope of the invention.

### EXAMPLES

The following examples are illustrative of the invention and should not be construed as limiting in any way the general nature of the disclosure of the description throughout this specification.

### Example 1

### Adhesion and Invasion of AIEC

Lactobacillus species L. parafarraginis Lp18, L. buchneri Lb23, L. rapi Lr24, L. zeae Lz26, L. paracasei Lp 9 and L. diolivorans Ld3 were tested for their ability to competitively inhibit adhesion and invasion (and translocation) of a pathogenic E. coli AIEC strain in the gut epithelium. The Lactobacillus strains were maintained at −80° C. in de Man, Rogosa, Sharpe (MRS) broth with 20% glycerol. They were grown on MRS agar as a working culture and regrown in MRS broth for 18 hours at 37° C. before each adhesion or invasion assay.

The AIEC strain (strain F44A-1) is a wild strain isolated from a patient with inflammatory bowel disease and contains all virulence genes associated with AIEC, and is consistent with AIEC diffuse adhesion pattern to Caco-2 cells as well as survival and replication in macrophages.

To assess the ability of each of the Lactobacillus species to inhibit interaction of these species with human gut epithelium, the cell lines Caco-2 (ATCC HTB-37) and HT29 (ATCC HTB-38) MTX representing gut epithelium were used. Coco-2 cells can be differentiated in culture medium to form a polarized cell monolayer with tight junctions and microvilli to resemble important characteristics of human intestinal mature enterocytes. HT29-MTX is mutated to produce mucin, similar to goblet cells of the gut epithelium. Cells were grown in 50 ml culture flasks to confluence in Eagle's Minimum Essential Medium, supplemented with 20% (v/v) Fetal bovine serum (FBS) for Caco-2 cells and 15% (v/v) FBS for HT29-MTX cells and 1% (v/v) penicillin-streptomycin. The cell cultures were maintained at 37° C. in an atmosphere of 5% carbon dioxide. Culture media were monitored closely and changed every 48 h. At confluence, cells were sub-cultured into the 8 well chamber slides for adhesion assays and sterile 96-well flat bottom plates for invasion assays.

### Adhesion Assays

Prior to the adhesion assay, cell lines were seeded onto 8 well glass chamber slide system. The cells were grown to ˜75% confluence, and prior to the assay, the medium was changed to antibiotic-free medium. Using 15ml tubes, AIEC isolates were cultured in LB broth for 4 hours, Lactobacilli strains were cultured in MRS broth overnight at 37° C. with agitation (140 strokes/min). Cell suspensions were centrifuged at 3,500 rpm for 12 minutes and the supernatant was discarded. The pellets were resuspended in phosphate-buffered saline (PBS) (pH 7.4), and 100 μl of the suspension (˜1.0×10⁹ cfu/ml, OD=1 at 600 nm) was inoculated into the appropriate chambers.

For the competitive adhesion, Lactobacilli and AIEC were inoculated together prior to incubation at 37° C. for 90 min For the ability of Lactobacilli to exclude AIEC, Lactobacillus suspension (1.0×10⁹ cfu/ml) were inoculated for 60 min before infecting with the same concentration of AIEC, then incubated at 37° C. for 90 min. After incubation, non-adherent bacteria cells were washed off with PBS (pH 7.4), cells were fixed with 95% ethanol (v/v) for 5 mins and stained using Gram stain to differentiate between Gram-positive Lactobacilli and Gram-negative AIEC and observed under a light microscope. Percentage of microbial adhesion to the cell lines were determined by counting cells showing adhesion on 100 randomly selected cells, while the number of adhering bacteria per cell was determined by counting the number of bacteria attached to 25 randomly selected cells.

### Invasion Assays

For the invasion assay, bacteria were grown as described in adhesion assay and inoculated onto Coco-2 and HT29-MTX cells grown to confluence in 96 well plates. Bacteria isolates were cultured for the invasion assays similar to the adhesion assay except that the concentration of bacteria was adjusted to (˜1×10⁸ cfu/ml). For competitive invasion, the Lactobacilli and the AIEC were inoculated at the same time followed by 2 hours incubation at 37° C. For the ability of Lactobacilli to inhibit/decrease the rate of AIEC invasion, the Lactobacilli were inoculated first and incubated for 60 min then AIEC was inoculated and incubated for another 2 hours at 37° C.

After the incubation, the monolayer was washed three times with PBS, then incubated in EMEM containing gentamicin (150 μg/ml) for 1 hour at 37° C., to kill non-invading cells. The monolayer was then washed 3 times with PBS and lysed with 0.1% (v/v) Triton X-100 to release the invading AIEC. The lysate was then serially diluted and 100 μl volumes were plated onto MacConkey Agar No 3 plates and incubated for 24 hours at 37° C. before colonies were counted. Mean±SEM of the colony forming units were calculated with due corrections for the dilution factors. E. coli strain 46-4 were used as negative control for both assays.

### Statistical Analysis

All experiments were carried out in triplicates. GraphPad Prism statistical software (Version 8.0.0) was used for statistical analysis. Two-way ANOVA followed by Tukey's multiple comparisons test were used to determine the differences in the mean level of adhesion and invasion among strains across all test groups. Relationships between adhesion and invasion capabilities of strains were evaluated using Pearson's correlation coefficient and differences were considered statistically significant if P<0.05.

### Results

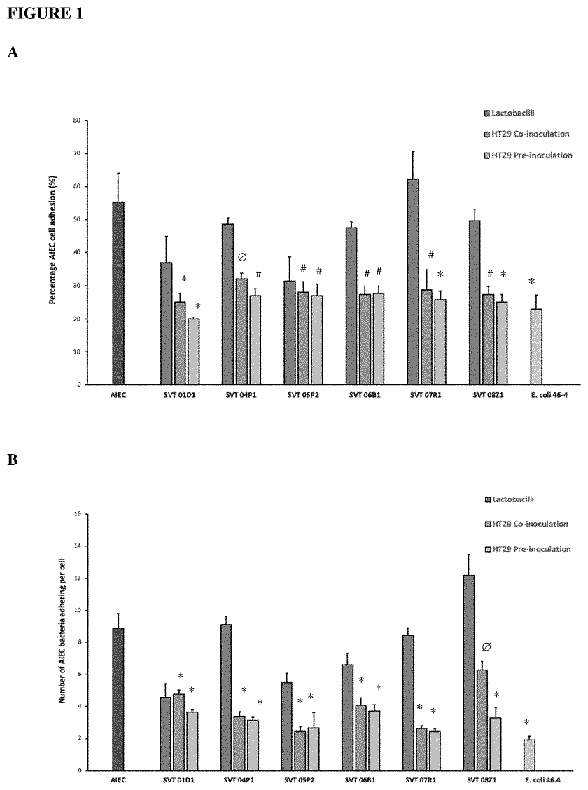

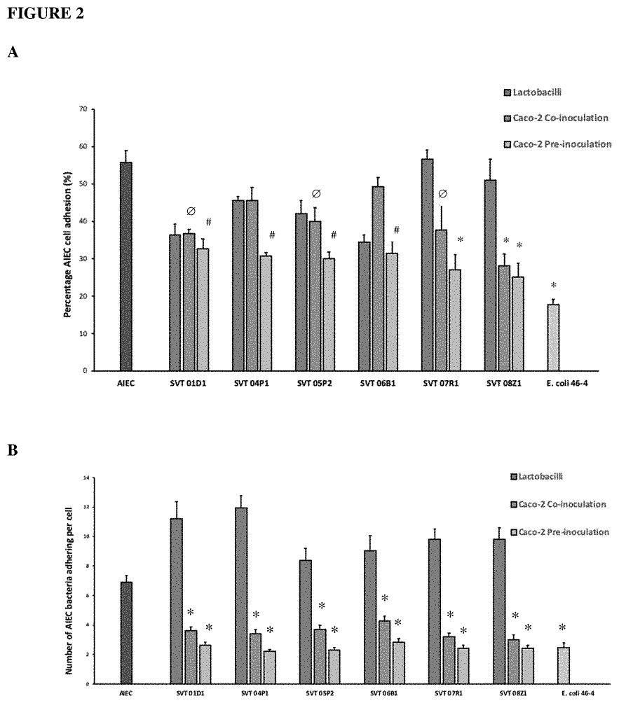

AIEC colonised 55% of the cells upon inoculation on both cell lines which was significantly more than Lactobacillus strains tested (32%-52%), except for L. rapi SVT-24. Both co- and pre-inoculation of Lactobacillus strains significantly decreased adhesion of AIEC to both HT29-MTX (FIG. 1) and Caco-2 cell lines (FIG. 2). In all instances, pre-inoculation reduced adhesion of AIEC to a greater extent than co-inoculation.

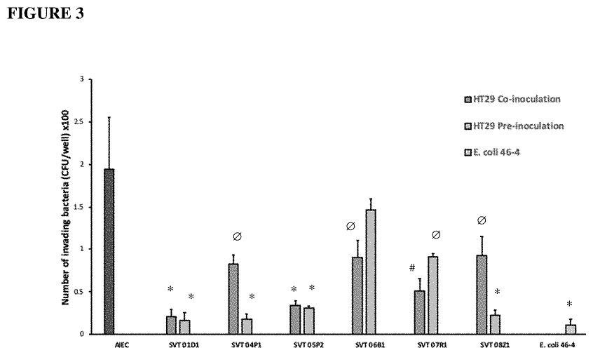

The ability of the Lactobacilli species to reduce invasion of the AIEC was also measured. Both co- and pre-inoculation of Lactobacillus strains significantly decreased invasion of AIEC in the HT29-MTX cell line with pre-inoculation in most instances reducing invasion to a greater extent except for SVT 06B1 (FIG. 3). There was no significant reduction in invasion of AIEC observed in the Caco-2 cell line with either co- or pre-inoculation (data not shown).

As demonstrated in this example, the six Lactobacillus species tested in this study interacted with two different gut epithelial cell lines to significantly reduce the adhesion and invasion of an AIEC. Pre-incubation of the cell lines with the Lactobacilli followed by the challenge with this pathogen was more favourable to competitively exclude the AIEC, suggesting the potential of these Lactobacilli to be used as prophylactic measures.

### Example 2

### Adhesion, Invasion and Translocation of Pathogenic E. Coli HMLN-1

Using the same assays and procedures as described in Example 1, the effect of the six Lactobacillus strains on adhesion and invasion of the pathogenic E. coli strain HMLN-1 was determined. The HMLN-1 strain was isolated from blood and mesenteric lymph nodes of a fatal case of a hospitalized patient and has been shown in many publications to be a professional translocator strain.

In addition, translocation was assessed. Specifically, a two-compartment model was used to assess the ability of HMLN-1 cells to translocate across Caco-2 and HT29-MTX cells in the presence of Lactobacillus strains. Cells were grown onto 24-well plates inserts with a porous membrane in EMEM, supplemented with 20% (v/v) FBS for Coco-2 cells and 15% (v/v) FBS for HT29-MTX cells, both combined with 1% (v/v) penicillin-streptomycin. Cell lines were grown to confluence and media replaced with antibiotic-free EMEM media prior to bacterial inoculation. Inhibition of HMLN-1 translocation was tested in the presence of Lactobacillus strains that were either co-inoculated with HMLN-1 or pre-inoculated prior to HMLN-1 infection. After incubation, 100 μL of EMEM was taken from the outer well and inoculated onto MacConkey agar plates that were incubated for 24 h at 37° C. and cells counted and expressed as mean±SEM.

E. coli JM109 strain was used as a negative control for the adhesion, invasion and translocation assays. Adhesion and invasion were conducted in triplicates and translocation was conducted in duplicates.

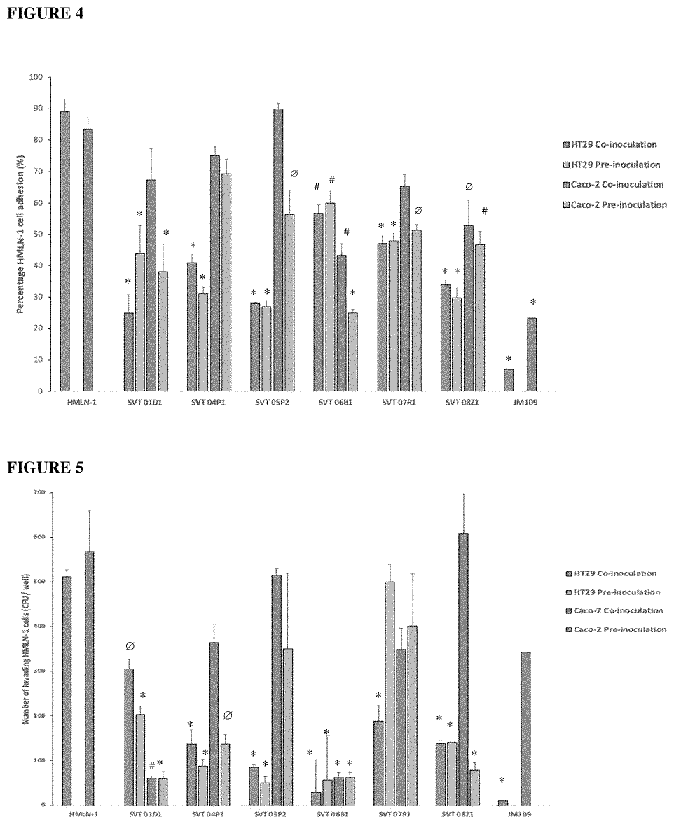

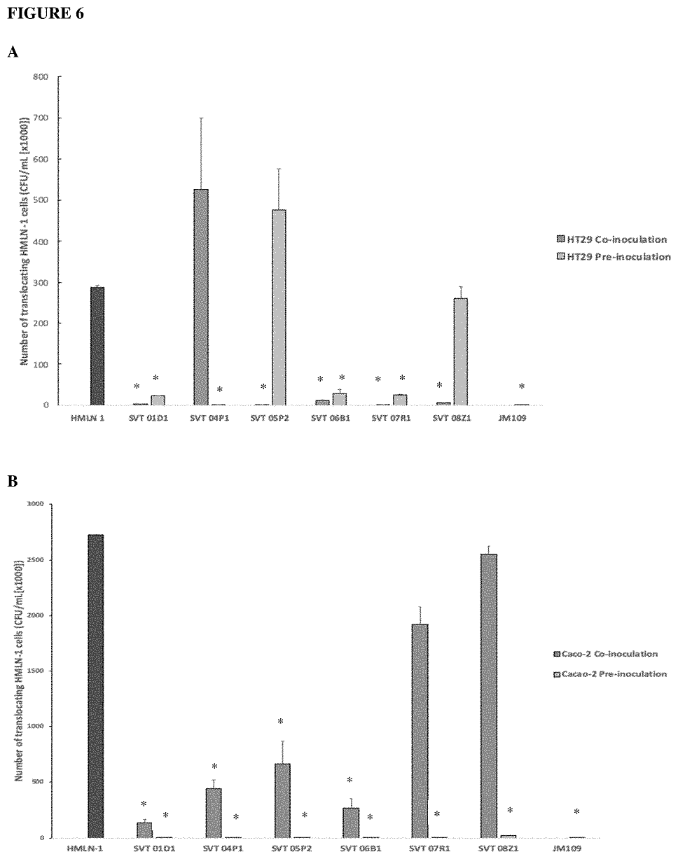

The percentage of HT29-MTX and Coco-2 cells showing adhesion of E. coli HMLN-1, alone or in the presence of Lactobacillus is shown in FIG. 4. Both co- and pre-inoculation with all Lactobacillus strains demonstrated a statistically significant reduction in the percentage of and number of HMLN-1 cells adhering to the HT29-MTX cell line. Both co- and pre-inoculation of Lactobacillus strains significantly decreased invasion of HMLN-1 in the HT29-MTX cell line (FIG. 5). There was a significant reduction in the translocation of HMLN-1 in both cell lines for both co- and pre-inoculation in most instances (FIG. 6).

### Example 3

### Adhesion and Invasion of Pathogenic E. Coli in an Improved Gut Epithelium Model

An improved gut epithelium model was developed by co-culturing Caco-2 and HT29-MTX cells at a ratio of 9 and 1, respectively. This model has many characteristics similar to the human gut epithelium. Lactobacillus species L. parafarraginis, L. buchneri, L. zeae, L. paracasei and L. diolivorans were tested for their ability to competitively inhibit adhesion and invasion (and translocation) of two pathogenic E. coli strains E. coli AIEC (F44A-1) and E. coli HMLN-1 in this model.

Experimental conditions were as described in Examples 1 and 2.

As a positive control the E. coli strains AIEC and HMLN-1 were incubated with the co-cultured Caco-2 and HT29-MTX cell lines and the level of adhesion and invasion was measured in CFU/mL. As a negative control, JM109 E. coli cells were incubated with the Caco-2/HT29-MTX cells in place of the AIEC and HMLN-1 strains. The Lactobacillus species tested were either co-incubated with the Caco-2/HT29-MTX cells along with the E. coli strains, or were pre-incubated with the Caco-2/HT29-MTX cells prior to the addition of the E. coli strains.

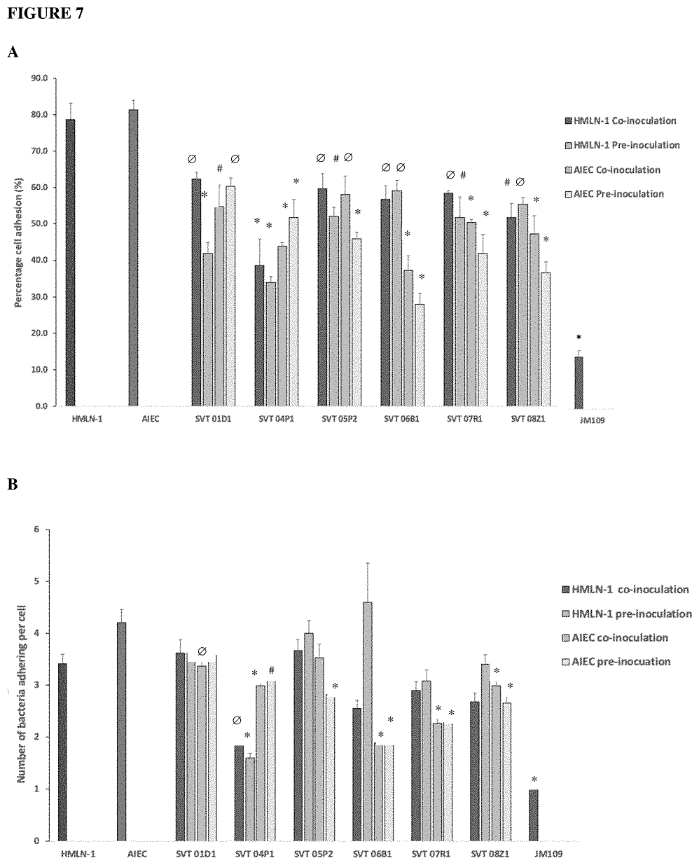

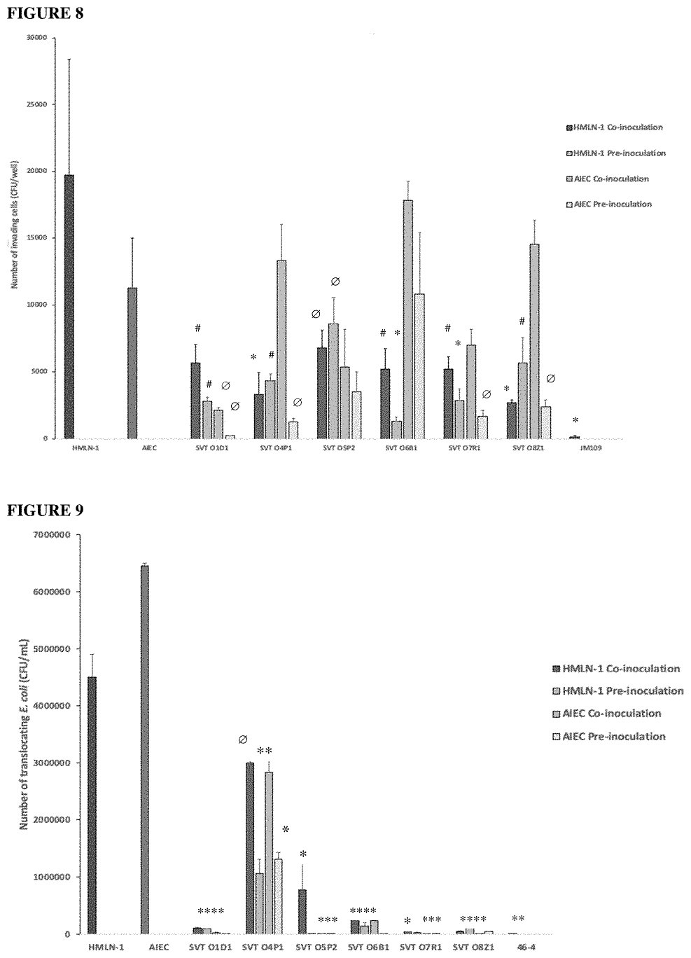

There was a statistically significant reduction of both HMLN-1 and AIEC percent adhesion on the co-culture cell line for all Lactobacillus strains when both co- and pre-inoculated (see FIG. 7AError! Reference source not found.) however, only SVT 04P1 demonstrated a statistically significant reduction of HMLN-1 and AEIC cell adhesion per co-culture cell for both co- and pre-inoculation (see FIG. 7B). There was a statistically significant reduction of HMLN-1 invasion when both co- and pre-inoculated with all Lactobacillus strains (see FIG. 8). There was a statistically significant reduction of translocation for both HMLN-1 and AIEC when co- and pre-inoculated with the Lactobacillus strains when compared to the positive controls (FIG. 9).

The results described in Examples 1, 2 and 3 show that the Lactobacillus strains investigated demonstrate good adherence ability to all gastrointestinal-like cell lines tested and statistically reduced adhesion of the pathogenic E. coli strains F44A-1 (AIEC) and HMLN-1 to all cell lines. The Lactobacillus strains significantly reduced invasion of AIEC on the HT29-MTX and co-culture cell lines; reduced invasion of HMLN-1 on all cell lines and significantly reduced translocation of both E. coli strains across all cell lines tested. The results suggest that these Lactobacillus strains have the therapeutic potential to reduce pathogenic E. coli infection in the human gastrointestinal tract and invasion into the blood stream.

### Example 4

### DSS-Induced Colitis Model

The inventors then examined the effect of L. paracasei (SVT04P1), L. buchneri (SVT06B1) and L. zeae (SVT08Z1) in a dextran sodium sulfate (DSS)-induced model of chronic colitis in mice. DSS was from MP BioMedicals, stored at room temperature. The model used in this study is a particularly effective model of severe, chronic colitis, based on the concentration of DSS employed (3%) and the administration of DSS over three 5-day cycles with two 7-day washout periods in between. Mice administered 3% DSS according to this regime show clear histopathological signs of ulceration, edema, inflammation and crypt loss in the colon (data not shown).

60 female C57BL/6NTac mice were divided into six treatment groups:
Group 1—non-treatment (negative control) group. n=10.
Group 2—3% DSS+vehicle (9% sterile saline). n=10.
Group 3—3% DSS+SVT04P1 at dose of 1.5×10¹⁰ cfu/ml. n=10.
Group 4—3% DSS+SVT06B1 at dose of 1.5×10¹⁰ cfu/ml. n=10.
Group 5—3% DSS+SVT08Z1 at dose of 1.5×10¹⁰ cfu/ml. n=10.
Group 6—3% DSS+combination of SVT04P1, SVT06B1 and SVT08Z1 at dose of 1.5×10¹⁰ cfu/ml. n=10.

Animals of Groups 2 to 6 received 3% DSS ad libitum via sterile drinking water on days 1 to 5, 13 to 17 and 25 to 29, while Group 1 animals continued to receive only sterile water as drinking water. Animals of Groups 2 to 6 also received vehicle or Lactobacillus (1.5×10¹⁰ cfu/ml) by oral gavage, from days 1 to 28, at a dose volume of 1 mL. On days where no DSS was provided to Groups 2 to 6, animals received sterile water. Lactobacillus were in sterile saline and 2-3% sucrose (stored at 4° C. until used). Prior to dosing, each bacterial formulation was analysed for cell viability/count. On day 1 of dosing 3% DSS solution was freshly prepared by dissolving DSS in sterile water.

Symptoms/characteristics of DSS-induced colitis (stool consistency and faecal blood occurrence) were assessed by measuring in-life endpoints from days 1 to 29, every other day. On days of dosing, evaluations were performed 1 to 2 hours after dosing.

Stool was collected from day 1 for each mouse and examined for consistency. Stool consistency was graded as follows: normal=0; soft, but still formed=1; very soft=2; diarrhoea=4. Faecal blood in the stool was detected using the Hemoccult Tape Test Kit (Beckman Coulter, according to manufacturer's instructions). Scoring for faecal blood was as follows: negative hemoccult=0; positive hemoccult (slight colour on strip)=1; positive hemoccult (darker colour on strip)=2; visible traces of blood=3; gross rectal bleeding=4. Percent body weight loss was also measured from day 1, graded as 0 (none), 1 (1-5%), 2 (>5-10%) and 3 (>10-20%).

Stool consistency scores, faecal occurrence scores and body weight loss scores were pooled to give a weighted in-life score of overall disease state, the disease activity index (DAI).

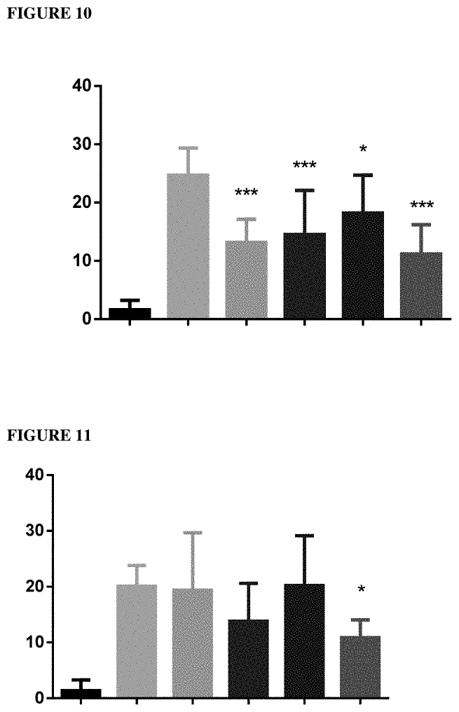

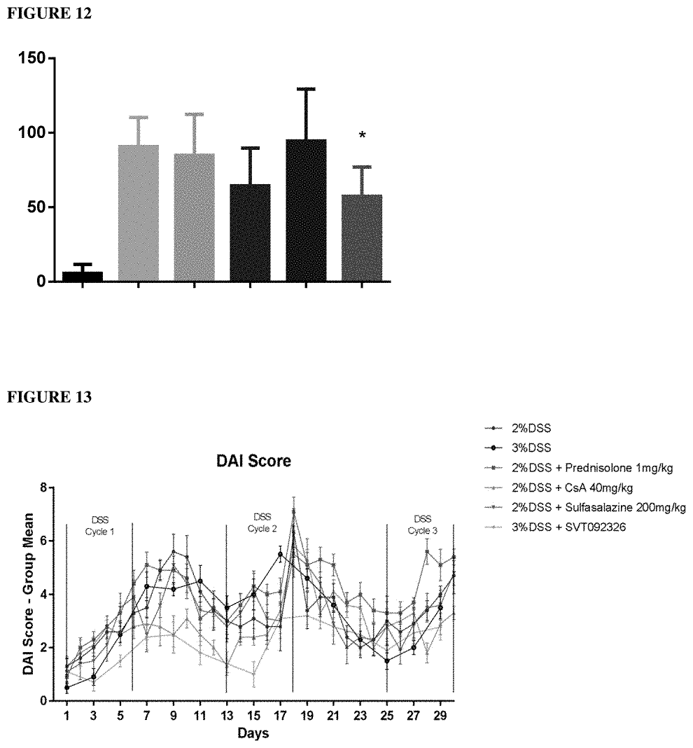

As shown in FIG. 10, stool consistency was significantly improved in mice of treatment groups 3 to 6 compared to group 2. Faecal blood occurrence was reduced in treatment groups 3, 4 and 6 compared to group 2, most significantly in treatment group 6, representing the combination of SVT-09, SVT-23 and SVT-26 (FIG. 11). The DAI was also improved in treatment groups 4 and 6 compared to group 2, again most significantly in the combination treatment group (group 6) (FIG. 12).

Mice were terminated at day 29. Terminal blood samples revealed a slight decrease in serum IL-6 concentration in groups 3, 5 and 6 compared to control group 2 and a decrease in KC/Gro (rodent equivalent of IL-8) in groups 3 and 5 compared to control group 2 (data not shown). There was also a trend of increasing colon length in each of treatment groups 3 to 6 compared to control group 2 as shown in Table 1.

TABLE 1
Colon length at termination

| Group | Colon length in mm (SEM) |
|---|---|
| Group 1 | 94.6 (1.34) |
| Group 2 | 67.8 (1.67) |
| Group 3 | 70.6 (1.34) |
| Group 4 | 70.7 (1.56) |
| Group 5 | 70.1 (2.15) |
| Group 6 | 72.9 (1.13) |

A number of therapeutic agents (including cyclosporin A, sulfasalazine and prednisolone) clinically employed in the treatment of inflammatory bowel diseases such as ulcerative colitis, have previously been tested in the same DSS-induced colitis model as used in the present study, by the same testing laboratory (Charles River Laboratories (CRL)). None of these drugs statistically improved the in-life disease score to the extent observed with the Lactobacillus treatments of the present study. For example, as shown herein, the DAI score for Group 6 was compared to previous data obtained by CRL that assessed efficacy of typical compounds used to clinically treat ulcerative colitis in a DSS murine model. Sulfasalazine, Prednisolone, 5-Aminosalicylic acid and 6-Thioguanine failed to demonstrate therapeutic efficacy, whereas only Cyclosporine A at 40 and 80 mg/kg showed some decrease in DAI score in the chronic DSS model, however not as efficacious as present Group 6 (FIG. 13). Data not shown for 5-Aminosalicylic acid and 6-Thioguanine. Importantly, the efficacy of the selected drugs was tested in a 2% DSS model, a less severe disease model than was used in the present study (3% DSS). The results obtained in the present study represent a significant advance in prospective treatment for ulcerative colitis when compared to existing therapies.

### Example 4A

A further combination of Lactobacillus strains was tested in the DSS-induced model of ulcerative colitis as described above:
Group 7—3% DSS+combination of SVT01D1, SVT05P2 and SVT06B1 at dose of 1.5×10¹⁰ cfu/ml. n=10

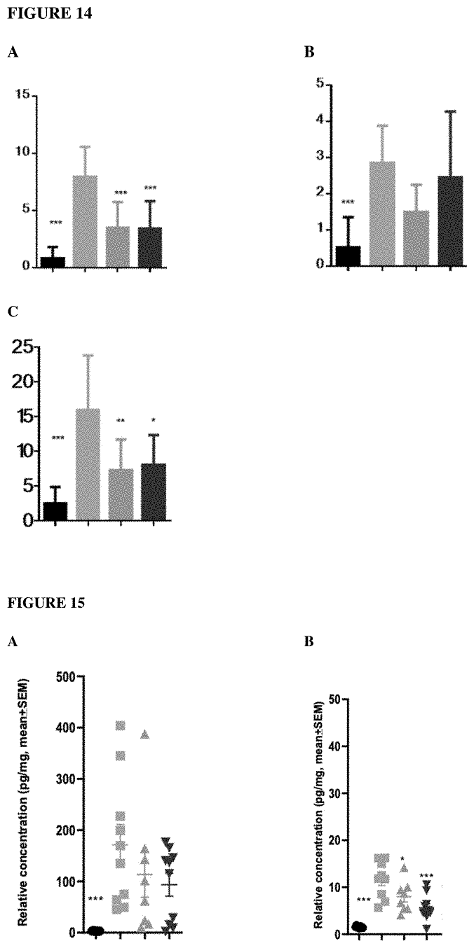

Faecal blood occurrence, stool consistency and DAI were determined for Group 7 as described above for Groups 1 to 6. Data is shown, in comparison to Groups 1, 2 and 6 (each as described in Example 4), in FIG. 14. Statistically significant reductions in faecal blood occurrence and improvements in DAI were observed.

Cytokine expression was also measured. Statistically significant increases in IL-6 and TNFα were observed in Group 2 compared to Group 1. Reductions in IL-6 expression were observed in Groups 6 and 7 compared to Group 2, and statistically significant reductions in TNFα expression were observed in Groups 6 and 7 compared to Group 2 (p<0.001 and p<0.05, respectively) (FIG. 15).

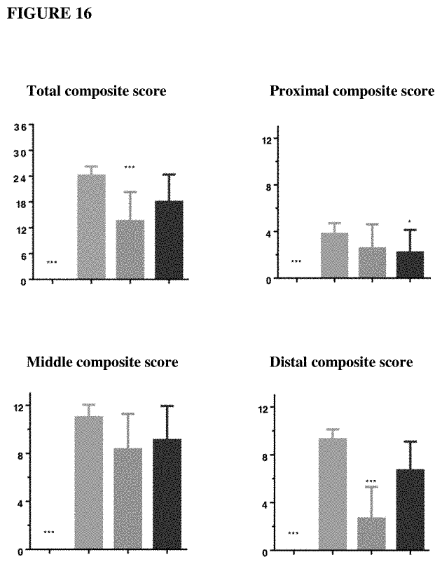

Colon samples were analysed for overall ulcer extent, percent of section affected by any inflammatory changes, percent of section affected by severe inflammatory changes with obliteration of normal architecture, erosion/ulceration and/or crypt abscesses and a total composite score calculated by the sum of the three individual scores for each colon segment. Scoring was calculated for each of the proximal, middle and distal sections of the colon samples. An overall total composite score demonstrated a statistically significant reduction in ulceration and inflammation for Group 7 compared to Group 2, and a statistically significant reduction in scoring was observed for the proximal segment in Group 6 compared to Group 2 and for the distal segment in Group 7 compared to Group 2 (see FIG. 16).

### Example 5

### Inhibition of Growth of Pathogens

The ability of the Lactobacillus species L. diolivorans, L. paracasei, L. parafarraginis, L. rapi and L. zeae deposited pursuant to the Budapest Treaty as described hereinbefore were tested for their ability to inhibit the growth of bacterial pathogens associated with inflammation and various inflammatory conditions. Pathogens tested for this purpose were Campylobacter jejuni (ATCC 33291), Helicobacter pylori (ATCC 700824), Clostridium difficile (ATCC 9689), Salmonella typhimurium (ATCC 29630), Yersinia enterocolitica (ATCC 23715), Citrobacter sp (ATCC 51378), Streptococcus pyogenes (ATCC 19615), Streptococcus mutans (ATCC 25175), Klebsiella oxytoca (ATCC 700324), and Proteus mirabilis (ATCC 25933). C. jejuni and H. pylori were grown under microaerobic conditions at 37° C. in 5% Columbia horse blood agar (CBA). S. typhimurium, Y. enterocolitica, Citrobacter sp., K. oxytoca and P. mirabilis were grown under aerobic conditions at 37° C. in nutrient agar. S. pyogenes and S. mutans were grown under aerobic conditions with 5% CO₂ at 37° C. in 5% CBA. C. difficile was grown under anaerobic conditions at 37° C. in 5% CBA.

Inhibition of pathogen growth was determined using an agar well diffusion assay. Cultures of the five test Lactobacillus species were grown in MRS broth for 24 hours at 37° C. Each pathogen was sub-cultured onto nutrient agar plates or 5% CBA plates and incubated under the appropriate conditions (as described above) prior to resuspension in DPBS to give a McFarland standard of 0.5 (equivalent to 10⁸ CFU/mL). Each 0.5 McFarland standardised culture was spread, using a swab, onto nutrient agar plates or CBA plates and allowed to soak in prior to making 9 mm wells. 100 μL of each test Lactobacillus, was then added to individual wells and the plates were incubated for 24 hours at 37° C. 5 μg ciprofloxacin was used as a positive control, and sterile MRS broth only was used as a negative control. Following incubation, zones of inhibition or zones of growth reduction (hazing of growth) were measured (mm) and recorded. Two readings were taken for each zone and three replicates were used.

As expected, sterile MRS broth only applied to the pathogen-containing plates yielded no zones of inhibition or zones of reduction. As shown in Table 1, most of the Lactobacillus species showed strong inhibitory activity against most of the pathogens tested. Zones of inhibition resulting from application of 5 μg ciprofloxacin are also shown in Table 1.

TABLE 2
Zones of inhibition/zones of growth reduction (mm).
Values are the mean of six experiments (two
readings from each of three replicates.

| | SVT-03 | SVT-09 | SVT-18 | SVT-24 | SVT-26 | Ciprofloxacin |
|---|---|---|---|---|---|---|
| C. jejuni | — | 21.0 | 12.8 | — | 19.8 | 30.2 |
| H. pylori | — | 21.7 | 13.5 | — | 22.5 | 31.7 |
| C. difficile | 11.8 | 19.7 | 13.7 | — | 19.3 | — |
| S. typhimurium | 14.2 | 20.5 | 15.3 | 15.2 | 23.2 | 49 |
| Y. enterocolitica | 16.0 | 19.7 | 14.3 | 15.8 | 22.2 | 44.2 |
| Citrobacter sp. | 13.3 | 16.8 | 13.8 | 13.5 | 18.0 | 47.0 |
| S. pyogenes | 12.7 | 16.7 | 10.2 | 12.8 | 18.3 | 30.2 |
| S. mutans | — | 18.3 | 10.7 | — | 19.2 | 28.5 |
| K. oxytoca | 15.0 | 16.7 | 13.0 | 12.3 | 16.5 | 41.2 |
| P. mirabilis | 15.7 | 18.5 | 13.5 | 14.7 | 21.2 | 44.2 |

The ability of L. diolivorans, L. paracasei, L. parafarraginis, L. rapi and L. zeae deposited pursuant to the Budapest Treaty as described hereinbefore were then tested for their ability to inhibit the growth of pathogenic bacteria Staphylococcus aureus (ATCC 29213 and ATCC 25923), Staphylococcus epidermidis (clinical isolate), Pseudomonas aeruginosa (ATCC 27853) and Escherichia coli K12. These pathogens were grown under aerobic conditions at 37° C. in nutrient agar. Inhibition of growth was determined using an agar well diffusion assay as described above. In addition, cell free supernatants derived from culture of the Lactobacillus species were also tested. Briefly, after 24 hours growth, cultures of Lactobacillus were centrifuged at 10,000×g and the supernatant filter sterilised through a 0.2 μm membrane. 100 μl of cell free supernatant was added to the wells containing pathogen. Results are shown in Tables 2 and 3 below.

TABLE 3
Zones of inhibition/zones of growth reduction (mm) using live cultures of
Lactobacilli. Values are the mean of six experiments (two readings from each
of three replicates). Readings include the well diameter of 9 mm

| | SVT-03 | SVT-09 | SVT-18 | SVT-24 | SVT-26 | Ciprofloxacin |
|---|---|---|---|---|---|---|
| S. aureus | — | 12.0 | 13.5 | 11.8 | 15.0 | 33.7 |
| (ATCC 29213) | | | | | | |
| S. aureus | — | 17.5 | 12.3 | 11.0 | 19.0 | 33.7 |
| (ATCC 25923) | | | | | | |
| S. epidermidis | — | 15.0 | 19.0 | 16.0 | 15.3 | — |
| P. aeruginosa | 10.8 | 19.0 | 13.2 | 13.2 | 19.7 | 39.2 |
| E. coli | — | 17.2 | 12.7 | 13.0 | 17.7 | 40.0 |

TABLE 4
Zones of inhibition/zones of growth reduction (mm) using cell free supernatants
derived from Lactobacilli cultures. Values are the mean of six experiments (two
readings from each of three replicates). Readings include the well diameter of 9 mm

| | SVT-03 | SVT-09 | SVT-18 | SVT-24 | SVT-26 | Ciprofloxacin |
|---|---|---|---|---|---|---|
| S. aureus | — | — | — | — | 11.8 | 33.7 |
| (ATCC 29213) | | | | | | |
| S. aureus | — | 13.5 | — | — | 17.0 | 33.7 |
| (ATCC 25923) | | | | | | |
| S. epidermidis | — | 14.5 | — | — | 14.0 | — |
| P. aeruginosa | 10.5 | 16.5 | 11.0 | 11.3 | 18.3 | 39.2 |
| E. coli | — | 17.8 | — | — | 16.0 | 40.0 |

### Example 6

A 45-year-old male had been experiencing periodic but significant jaw pain as a result of temporomandibular joint disorder. The subject placed 2 mL of a liquid formulation containing Lactobacillus buchneri Lb23 under the tongue before swallowing on a daily basis. The formulation comprised about 10⁶ to 10⁸ CFU/mL of the microbial strain in sterile saline and 2-3% sucrose (stored at 4° C. until used). After 4 to 5 days the pain had diminished, and no further discomfort was experienced.

### Example 7

A 61-year-old female had complained of ongoing bowel problems including irritation, pain, bloating and inflammation of the colon and rectum. The subject orally ingested 2 mL of a liquid formulation containing Lactobacillus zeae Lz26 on a daily basis. The formulation comprised about 10⁶ to 10⁸ CFU/mL of the microbial strain in sterile saline and 2-3% sucrose (stored at 4° C. until used). After 3 to 4 days the bowel problems had improved and all symptoms were reduced. The subject continued to use the formulation and found minimal digestive problems were experienced while taking the treatment.

### Example 8

A 19-year-old male has been suffering from moderate to severe facial acne for a number of years with only mild success using topical creams and antibiotics. The subject rubbed about 1 mL daily over the facial area using a liquid formulation containing Lactobacillus paracasei Lp9. The formulation comprised about 10⁶ to 10⁸ CFU/mL of the microbial strain in sterile saline and 2-3% sucrose (stored at 4° C. until used). After one week the number and size of pimples had decreased and in particular the pain associated with swollen and inflamed areas was greatly reduced. The subject commented that the blemished and painful areas were much less noticeable during treatment.

### Example 9

A 48-year-old male suffered from pain in the right knee due to many years spent playing competitive and social football and tennis. The pain was infrequent but always occurred after heavy use of the joint and was accompanied with significant inflammation. Each time the inflammation and pain occurred the subject placed 2 ml of a liquid formulation comprising a combination of Lactobacillus buchneri Lb23, Lactobacillus zeae Lz26 and Lactobacillus paracasei Lp9 under the tongue for several minutes before swallowing, on a daily basis. The formulation comprised about 10⁶ to 10⁸ CFU/mL of the microbial strains in sterile saline and 2-3% sucrose (stored at 4° C. until used). Within 2-3 days the pain and swelling around the knee begins to decrease and the subject can usually walk and move the knee without any major pain or discomfort.

### Example 10

A 56-year-old male was experiencing significant gastrointestinal distress for several years, coinciding with extensive travel to the South-East Asia region. The subject was afflicted with recurring abdominal cramping, occasional nausea, and a Bristol stool score of 6-7. A treatment regimen was established, comprising twice-daily doses of 2.5 mL of a 1×10⁷ CFU/mL liquid formulation of Lactobacillus paracasei Lp9. Three days post treatment, the subject reported a marked improvement in gastrointestinal symptoms. Buoyed by the improvement, the subject elected to include a once-daily 5 mL dose of 2.5×10⁹ Lactobacillus buchneri Lb23 in the regimen. After 2 weeks of treatment, the subject's Bristol stool score had improved to 4, with negligible reported gastrointestinal upset.

### Example 11

A 62-year-old female was suffering from moderate osteoarthritis, predominately in the knees and joints of the hand. The management approach recommended by her physician was paracetamol as required. However, the subject was still experiencing significant stiffness and loss of mobility, particularly of the fingers. A liquid formulation of 5×10⁹ CFU/mL of Lactobacillus parafarraginis Lp18 was taken orally. The dosage regimen was 5 mL, taken once daily. One week into the treatment, the subject noted a discernible improvement in the pain and swelling of affected joints. The treatment regimen was escalated to a 10 mL liquid formulation of Lactobacillus diolivorans Ld03, Lactobacillus zeae Lz26 and Lactobacillus parafarraginis Lp18, at 1×10⁹ CFU/mL. After a month of combination treatment, the subject was experiencing significantly less joint stiffness, reported substantial restoration of finger mobility, and decreased pain in the knee joints.

### Example 12

An 80-year-old male was suffering from severe osteoarthritis of the knee joints, resulting in a loss of mobility and a deterioration in quality of life. The subject had been managing his condition using non-steroidal anti-inflammatory drugs (NSAIDs), under the direction of a physician. A treatment of 15 mL 1×10⁹ CFU/mL Lactobacillus parafarraginis Lp18 in a liquid formulation, taken orally once-daily was trialled. After four weeks of the treatment, the subject experienced less pain and swelling of the knees and was able to take short walks again. The subject included a secondary 15 mL 2.5×10⁷ CFU/mL daily dose of a combined formulation; Lactobacillus parafarraginis Lp18, Lactobacillus zeae Lz26, and Lactobacillus rapi Lr24. Two weeks of the combined treatment yielded continued improvement and the subject was able to return to light gardening several times a week.

### Example 13

A 25-year-old female suffered debilitating mastitis with significant pain and swelling, and to her distress, was unable to breast feed her newborn child. The subject took 5 mL of a liquid formulation containing 1×10⁸ CFU/mL of Lactobacillus paracasei Lp9 per day by mouth in the morning for 7 consecutive days. Her symptoms improved, such that she increased the dose to 10 mL per day for a further 7 days, and noticed further improvement in her condition. Following the two-week treatment, the subject felt her mastitis was significantly improved, and further, to her delight, she was able to resume breast feeding her newborn.

### Example 14

A 35-year-old female afflicted with rheumatoid arthritis on a treatment regime of 20 mg methotrexate weekly, suffered frequent flare-ups with swelling in a number of her proximal interphalangeal joints. In addition to her existing treatment, the subject took 10 mL of a liquid formulation containing 1×10⁹ CFU/mL Lactobacillus zeae Lz26 twice daily by mouth for several weeks. After approximately two weeks, the subject noticed a reduction in pain and swelling in the currently affected joints, and following a month of treatment noted no additional flare ups. The subject felt the adjunct microbial treatment helped to stabilise her condition.

### Example 15

A 27-year-old female that reported chronic abdominal pain and discomfort for many years was given a liquid formulation comprising of Lactobacillus buchneri Lb23. The formulation constituted about 10⁶ to 10⁸ cfu/ml of the microbial strains in sterile saline and 2-3% sucrose (stored at 4° C. until used). After taking 10 ml every morning while having breakfast for 7 consecutive days, she reported a significant improvement in her symptoms after day 4. She also reported a pain free day on day 7.

### Example 16

A 19-year-old male returning from a 4-week road trip in Thailand reported recurring episodes of diarrhoea upon his return home. A liquid formulation comprising a combination of Lactobacillus buchneri Lb23, Lactobacillus zeae Lz26 and Lactobacillus paracasei Lp9 at 10⁶ to 10⁸ cfu/ml of the microbial strains in sterile saline and 2-3% sucrose (stored at 4° C. until used) was provided. He took 3 ml three times a day and observed an improvement of his conditions on day 2. He fully recovered on day 4.

### Example 17

A 55-year-old woman suffering from food poisoning after eating at her local fish and chips was given a single dose (35 ml), of a liquid formulation of Lactobacillus diolivorans Ld03 formulated at about 10⁸ cfu/ml in sterile saline and 2-3% sucrose (stored at 4° C. until used). She reported a significant improvement in her symptoms 10 hours post treatment and a back to normal digestive transit the next day.

### Example 18

An 18 year-old male reported experiencing moderate acne vulgaris for approximately one year, reporting to have 50-80 lesions (combination of comedones [˜60] and inflammatory lesions [˜20]) at any one time that were quite red and painful. He had attempted to reduce the lesion numbers with the use of non-prescription acne face washes, creams and gels but with little success with his skin becoming dryer. He first started applying 1 mL of Lactobacillus paracasei Lp9 at a concentration of 1×10⁸ cfu/mL via spray application (5 sprays per mL in a carrier of saline/sucrose) that enabled him to cover the entire surface of his face. He applied the bacteria twice daily, after cleansing, and started to notice a reduction in both inflammatory lesions and comedones after 7 days with a noticeable reduction in redness and pain. He reported approximately 10 inflammatory lesions and 40 comedones at day 7, that further reduced at day 14 to 5 inflammatory lesions and 25 comedones. After day 14 he added Lactobacillus buchneri Lb23 and Lactobacillus zeae Lz26 at 1×10⁸ cfu/mL each to the L. paracasei and continued to apply the spray (1 mL per application) twice daily for a further 14 days. At day 28 he reported the presence of 5 comedones and no inflammatory lesions. During the 28-day period he had not used any acne creams or gels and washed only with his usual face wash.

### Example 19

A 23 year-old female reported experiencing moderate acne since the age of 15 with an increasing severity of symptoms. She was diagnosed by her gynaecologist with endometriosis with irregular and painful periods. She had been taking the oral contraceptive pill for 4 years but reported no improvement in her acne symptoms. She had taken a course of tetracyclines at the recommendation of her general practitioner and while noticing an improvement in her skin while taking the antibiotics for 14 days per course, her acne symptoms reappeared once she completed the course. Furthermore, she experienced moderate gastrointestinal disturbances such as bloating and cramping with diarrhoea, which prevented her from continuing with such treatment. she initially began to apply 1 mL of Lactobacillus zeae Lz26 (1×10⁹ cfu/mL) twice per day after cleansing, in a carrier of saline/sucrose, spraying a total of 5 sprays (1 mL) to cover the surface of her face. Prior to treatment she reported approximately 80 comedones (opened and closed) and 30 inflammatory lesions, that were red and painful. After 7 days application she noticed a reduced lesion count with approximately 50 comedones and 20 lesions. She continued to report improvement in her skin each week while continuing to apply Lz26 for a further 49 days (8 weeks total). By day 56 she reported minimal comedones (10) and no inflammatory lesions.

### Example 20

A 45 year-old male had been experiencing recurrent tinea (athlete's foot) between the toes, principally between the middle, fourth and little toe on each foot. The subject placed ˜1 mL of a liquid formulation containing Lactobacillus buchneri Lb23 on the surface of the infected areas daily for three consecutive days. The formulation comprised about 10⁶ to 10⁸ CFU/mL of the microbial strain in sterile saline and 2-3% sucrose (stored at 4° C. until used). After 3 days symptoms had improved and after 5 days fungal infection had disappeared.

### Example 21

A 44 year-old male had a ringworm fungal infection on his lower back which was as a result of training on a communal impact mat at the gym. The subject placed ˜1 mL of a liquid formulation containing Lactobacillus zeae Lz26 on the surface of the infected areas daily for three consecutive days. The formulation comprised about approximately 1×10⁸ cfu/mL of the microbial strain in sterile saline and 2-3% sucrose (stored at 4° C. until used). After 5 days the fungal infection had disappeared.

### Example 22

A 44 year-old female had signs of early stage toenail fungus appearing in the nail on her right foot big toe. The subject placed ˜1 mL of a liquid formulation containing Lactobacillus zeae Lz26 on the surface of the infected areas daily for five consecutive days. The formulation comprised about approximately 1×10⁸ cfu/mL of the microbial strain in sterile saline and 2-3% sucrose (stored at 4° C. until used). After 5 days the immediate signs of the fungal infection had disappeared and the nail was able to heal naturally over time and normal growth.

### Example 23

A 48-year-old male, diagnosed with atopic dermatitis (AD) since the age of six months old, which was on average of moderate severity, occasionally becoming severe in times of stress. The dermatitis lesions were extremely dry, red and very itching (pruritus) causing enough discomfort that significantly impacted on his sleep quality. He is currently under the medical care of a specialist dermatologist who has prescribed topical corticosteroids to treat acute inflammatory episodes, particularly on both antecubital fossa's and popliteal fossa's and sometimes on his torso. The AD affects approximately 30% of his body. He has used the topical corticosteroids intermittently since childhood. He has had extensive courses of antibiotics (both oral and topical) to treat persistent Staphylococcus aureus infections. His daily regimen includes cleansing with soap free QV cleanser and twice daily application of QV Flare up cream to his torso and extremities. Regardless of his adherence to recommended medical treatments, he continued to experience moderate symptoms, particularly when he experienced stressful episodes at work and the hot humid conditions in spring and summer He proceeded to apply a formulation containing Lactobacillus paracasei Lp9 in a saline/sucrose carrier twice daily, with each 0.2 mL spray covering an area approximately 20 cm² and the total area treated at each application was up to 200 cm². Each 0.2 mL spray contained 1×10⁹ CFU. During this time, he refrained from applying the QV cream to the treated areas. After 5 days of treatment, he noticed a marked improvement in the severity of his lesions with the redness and pruritus reduced by approximately 30% and he had noticed his sleep quality had improved due to decreased scratching. After a further 14 days of applying the L. paracasei Lp9, he had reported significant improvement in the condition of his skin and rated the severity of his AD lesions as very mild with no overt signs of S. aureus infection. He had experienced a reduction in lesion redness, dryness and pruritus by up to 80%.

### Example 24

A 50 year-old-female diagnosed with Psoriasis at the age of 30 years. Plaques of intense red scaly skin predominantly affects her hairline, nape of the neck and behind the ears, with patches affecting her elbows. She often uses topical corticosteroids to reduce inflammatory episodes, has experienced numerous Staphylococcus aureus infections that have required both topical and oral antibiotics. She usually applies QV cream to the affected areas. She proceeded to apply Lactobacillus buchneri Lb23 to the plaques on her hairline, nape of her neck and behind her ears, twice daily at a dose of 1×10⁹ cfu/0.2 mL spray, with each spray covering an area of 20 cm². She applied 8 sprays in total twice per day. At the end of day 7, she noticed that the plaques behind her ears had completely disappeared whilst the plaques on her hairline and neck reduced in severity by about 50%. She continued to apply to L. buchneri Lb23 for a further 14 days at which time all plaques had disappeared with residual redness only.

### Example 25

A 39 year-old female had suffered from a variety of health complications that were linked to excessive Streptoccoccus bacteria, including recurrent Strep throat, tonsillitis, halitosis and gastro-intestinal complaints. The subject swallowed ˜1-2 mL of a liquid formulation containing Lactobacillus zeae Lz26 daily for five consecutive days and then every second day for two weeks the subject placed 2 ml of a liquid formulation comprising a combination of Lactobacillus buchneri Lb23, Lactobacillus zeae Lz26 and Lactobacillus paracasei Lp9 under the tongue for several minutes before swallowing. The formulation comprised about approximately 10⁸ cfu/mL of the microbial strain(s) in sterile saline and 2-3% sucrose (stored at 4° C. until used). The subject reported a reduction in throat soreness, improved halitosis and improved gastrointestinal function during the period of treatment.

### Example 26

A 45 year old female was diagnosed with a Helicobacter pylori infection. Prior to beginning treatment with a combination of Lactobacillus paracasei Lp9 and Lactobacillus zeae Lz26, a ¹⁴C urea breath test conducted on the subject returned a result of 1356 disintegrations per minute (dpm), a highly positive result for the presence of H. pylori in the stomach. The subject began a course of treatment by oral administration of 10 ml Lactobacillus paracasei Lp9 and Lactobacillus zeae Lz26 at a total concentration of 2×10⁸ cfu/mL daily for one month. At the end of this period, a ¹⁴C urea breath test returned a result of 654 dpm, a reduction of more than 50%.

### Deposit Details

Details of the biological material deposited pursuant to the Budapest Treaty are provided hereinbefore in the specification. In summary:

Lactobacillus parafarraginis SVT-18 was deposited pursuant to the Budapest Treaty with the Belgium Co-Ordinated Collection of Micro-organisms (BCCM), Belgian Coordinated Collections of Microorganisms (BCCM) Federal Public Planning Service Science Policy, 8, rue de la Science B-1000, Brussels, Belgium, on 27 Feb. 2019 under Accession Number LMG P-31292.

Lactobacillus buchneri SVT-23 was deposited pursuant to the Budapest Treaty with the Belgium Co-Ordinated Collection of Micro-organisms (BCCM), Belgian Coordinated Collections of Microorganisms (BCCM) Federal Public Planning Service Science Policy, 8, rue de la Science B-1000, Brussels, Belgium, on 27 Feb. 2019 under Accession Number LMG P-31293.

Lactobacillus zeae SVT-26 was deposited pursuant to the Budapest Treaty with the Belgium Co-Ordinated Collection of Micro-organisms (BCCM), Belgian Coordinated Collections of Microorganisms (BCCM) Federal Public Planning Service Science Policy, 8, rue de la Science B-1000, Brussels, Belgium, on 27 Feb. 2019 under Accession Number LMG P-31295.

L. rapi SVT-24 was deposited pursuant to the Budapest Treaty with the Belgium Co-Ordinated Collection of Micro-organisms (BCCM), Belgian Coordinated Collections of Microorganisms (BCCM) Federal Public Planning Service Science Policy, 8, rue de la Science B-1000, Brussels, Belgium, on 27 Feb. 2019 under Accession Number LMG P-31294.

Lactobacillus paracasei SVT-09 was deposited pursuant to the Budapest Treaty with the Belgium Co-Ordinated Collection of Micro-organisms (BCCM), Belgian Coordinated Collections of Microorganisms (BCCM) Federal Public Planning Service Science Policy, 8, rue de la Science B-1000, Brussels, Belgium, on 27 Feb. 2019 under Accession Number LMG P-31290.

Lactobacillus diolivorans SVT-03 was deposited pursuant to the Budapest Treaty with the Belgium Co-Ordinated Collection of Micro-organisms (BCCM), Belgian Coordinated Collections of Microorganisms (BCCM) Federal Public Planning Service Science Policy, 8, rue de la Science B-1000, Brussels, Belgium, on 27 Feb. 2019 under Accession Number LMG P-31287.

1. A method for treating or preventing inflammation, or an inflammatory or autoimmune condition, or one or more symptoms associated therewith, in a subject, comprising administering to the subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

2. The method of claim 1, wherein the inflammation is inflammation of the gastrointestinal tract, urinary tract skin, nails or joints or wherein the inflammatory or autoimmune condition is an inflammatory or autoimmune condition of the gastrointestinal tract, urinary tract, skin, nails or joints.

3. The method of claim 1, wherein the inflammation is associated with food poisoning, diarrhoea, gastric ulcers, mouth ulcers, dental caries or periodontal disease.

4. (canceled)

5. The method of claim 1, wherein the inflammation, inflammatory condition or autoimmune condition is caused or induced by, or otherwise associated with, a bacterial infection, a fungal infection, a viral infection or a parasitic infection.

6. (canceled)

7. (canceled)

8. The method of claim 2, wherein the gastrointestinal inflammation or condition of the gastrointestinal tract is, or is associated with, gastritis, gastroenteritis or an inflammatory bowel disease.

9. The method of claim 8, wherein the condition is irritable bowel syndrome.

10. The method of claim 8, wherein the inflammatory bowel disease is colitis.

11. (canceled)

12. The method of claim 10, wherein the ulcerative colitis is chronic ulcerative colitis.

13. The method of claim 8, wherein the subject is administered a combination of L. paracasei, L. buchneri and L. zeae or a combination of L. diolivorans, L. parafarraginis and L. buchneri.

14. The method of claim 2, wherein the gastrointestinal inflammation or condition of the gastrointestinal tract is, or is associated with, gingivitis or pharyngitis.

15. The method of claim 2, wherein the condition of the urinary tract is, or is associated with, cystitis, urethritis, pyelonephritis, asymptomatic bacteriuria or a catheter-associated urinary tract infection.

16. The method of claim 2, wherein the condition of the skin or nails is, or is associated with, psoriasis, dermatitis, eczema, rosacea, acne, ichtyosis, tinea or other skin or nail condition characterized by or associated with one or more of inflammation, plaques, skin lesions and/or infection.

17. The method of claim 2, wherein the condition of the joints is, or is associated with, arthritis.

18. The method of claim 17, wherein the arthritis is rheumatoid arthritis or osteoarthritis.

19. A method for treating or preventing a bacterial infection of the gastrointestinal tract, urinary tract, skin, nails or joints, comprising administering to a subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

20. The method of claim 19, wherein the bacterial infection causes, induces and/or is otherwise associated with inflammation, or an inflammatory or autoimmune condition.

21. The method of claim 19, wherein the bacterial infection is associated with adhesion and/or invasion of the gastrointestinal epithelium by bacteria causing the infection.

22. (canceled)

23. A method for inhibiting and/or preventing adhesion of a bacterial pathogen to the gastrointestinal mucosa and/or invasion by the pathogen of gastrointestinal epithelial cells in a subject, comprising administering to the subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

24. (canceled)

25. The method of claim 23, wherein the bacterial pathogen is a pathogen that colonises the lower gastrointestinal tract.

26. (canceled)

27. A method for promoting wound healing in a subject, comprising administering to the subject a Lactobacillus species selected from Lactobacillus buchneri, Lactobacillus zeae, Lactobacillus rapi, Lactobacillus paracasei, Lactobacillus parafarraginis, and Lactobacillus diolivorans, and/or a culture supernatant or cell free filtrate derived from culture media in which the Lactobacillus has been cultured.

28. (canceled)

29. (canceled)

30. (canceled)
